(19) United States
(12) Patent Application Publication
(10) Pub. No.: US 2024/0290455 A1
(43) Pub. Date: Aug. 29, 2024

(54) BIOMARKER-SEARCHING DEVICES AND METHOD THAT CAN PREDICT EFFECTIVENESS AND OVERALL SURVIVAL OF ICI TREATMENT FOR CANCER PATIENTS USING NETWORK-BASED MACHINE LEARNING TECHNIQUES

(71) Applicants: POSTECH RESEARCH AND BUSINESS DEVELOPMENT FOUNDATION, Pohang-si (KR); IMMUNOBIOME INC., Pohang-si (KR)

(72) Inventors: Sang Uk KIM, Pohang-si (KR); Jung Ho KONG, Busan (KR); In Hae KIM, Pohang-si (KR); Chang Wook Park, Seoul (KR)

(21) Appl. No.: 18/631,165

(22) Filed: Apr. 10, 2024

Related U.S. Application Data
(63) Continuation of application No. PCT/KR2022/014088, filed on Sep. 21, 2022.

(30) Foreign Application Priority Data
Oct. 12, 2021 (KR) 10-2021-0135020
Mar. 31, 2022 (KR) 10-2022-0040238

(51) Int. Cl. G16H 20/17; G16B 20/30; G16H 50/70

(52) U.S. Cl. CPC G16H 20/17; G16B 20/30; G16H 50/70

(57) ABSTRACT

The present disclosure is to provide a biomarker-searching method that can predict responses to ICI treatment and overall survival of ICI-treated patients. When the device and the method according to the present disclosure are used, it is possible to detect a biomarker capable of accurately predicting effectiveness of ICI treatment on cancer patients and overall survival of the cancer patients. Accordingly, it is possible to maximize the effectiveness of ICI treatment.

### STATEMENT DESIGNATING GRACE PERIOD INVENTOR DISCLOSURES

Applicant informs that the subject matter of this patent application was disclosed by the inventor or by another who obtained the subject matter disclosed directly or indirectly from the inventor or joint inventor one year or less than before the effective filing date of a claimed invention which does not quality as prior art under 35 U.S.C. 102(b)(1), as follows: JungHo Kong et al., Network-based machine learning in colorectal and bladder organoid models predicts anti-cancer drug efficacy in drugs, Nature Communications, Vol. 11, Thesis No. 5485 (page 1-13), Oct. 30, 2020.

### TECHNICAL FIELD

The present invention relates to biomarker-searching devices and method that can predict effectiveness and overall survival of ICI treatment for cancer patients using network-based machine learning techniques.

### BACKGROUND

Cancer accounts for the highest death rate in Korea, and there is a continuous demand for development of anti-cancer agents.

In the process of developing of anti-cancer drug, chemical drugs for killing cells using the characteristics of rapidly proliferating cancer cells, and for attacking specific molecule or signaling system existed, but they had several side effects. Then, immune anticancer drugs that could minimize side effects using innate immunity in the body appeared.

Cancer immunotherapy refers to a cancer therapy approach or method of activating the immune system of a human body to cause the immune system to combat cancer cells. In the cancer immunotherapy, only cancer cells are attacked using the immune system, resulting in less side effects than existing anti-cancer treatments, and the memory and adaptiveness of the immune system are used, enabling long-term anti-cancer efficacy. The cancer immunotherapy that overcomes drawbacks of existing anti-cancer agents as described above has been receiving a lot of attention as a new paradigm in cancer treatment, and Science Magazine chose cancer immunotherapy as the research of the year in 2013.

Immuno-oncology drug can be categorized into a therapeutic antibody (Rituximab, etc.) for targeting a tumor antigen, an immune checkpoint inhibitor for reactivating an immune cell, and an immune cell therapy for directly administering an immune cell.

Over the past several years, immune checkpoint inhibitors (ICIs) have drastically improved the clinical treatment of cancer patients. In clinical trials, using ICIs generally induced fewer side effects than chemotherapy with longer-lasting treatment benefits. Accordingly, the use of ICIs has expanded to a constantly growing list of cancer types, including melanoma, bladder cancer, and gastro-esophageal cancer.

However, despite the clinical benefits gained from ICI treatments, one major limitation is that only a few patients respond to immunotherapy (˜30% in solid tumors), and toxicity may occur after ICI treatment. Therefore, a method must be developed to identify biomarkers that can detect immunotherapy responders before drug administration, providing information about the clinical use of ICIs and improving the survival of cancer patients.

A major challenge of precision medicine using immunotherapy is identifying markers from immunotherapy-treated patients that can robustly predict drug responses across multiple cancer patient cohorts. For example, programmed cell death 1 (PD1)/programmed cell death-ligand 1 (PD-L1) expression by immunohistochemistry is a Food and Drug Administration (FDA)-approved companion diagnostic test for various cancer types. Accordingly, many studies have reported a positive correlation between PD-L1 expression and the ICI response in non-small cell lung cancer. Strikingly, however, other studies have reported no significant correlation between PD-L1 expression and the ICI treatment response, and some studies have even revealed that ICI responders display low PD-L1 expression levels. These inconsistent predictions of previously identified biomarkers necessitate identifying new biomarkers that robustly predict the immunotherapy response. Litchfield et al. recently found that conventional biomarkers can explain only ˜60% of the ICI response, suggesting that novel factors are yet to be discovered (Litchfield, K. et al. Meta-analysis of tumor- and T cell-intrinsic mechanisms of sensitization to checkpoint inhibition. Cell 184, (2021).).

Network biology offers a powerful means to identify robust biomarkers. These network-based approaches exploit observations that genes with similar phenotypic roles tend to co-localize in a specific region of a Protein-Protein Interaction (PPI) network. This tendency has been leveraged to identify gene modules that are much more robust in predicting phenotypic outcomes than using single gene-based approaches. For example, Hofree et al showed that patients with somatic mutations in similar network regions displayed similar clinical outcomes, although many clinically identical patients share no more than a single mutation (Hofree, M., Shen, J. P., Carter, H., Gross, A. & Ideker, T. Network-based stratification of tumor mutations. Nat. Methods 10, 1108-1115 (2013)). Furthermore, Guney et al. demonstrated that a drug's efficacy can be inferred from the proximity between drug targets and disease genes (Guney, E., Menche, J., Vidal, M. & Barabasi, A.-L. Network-based in silico drug efficacy screening. Nat. Commun. 7, 10331 (2016).). In addition, it has previously reported that drug response biomarkers that predict the overall survival in cancer patients can be identified via network proximity using the pharmacogenomics data of patient-derived organoid models. Altogether, evidence indicates that the network-based approach provides predictive and less noisy biomarkers, but the usefulness of the approach has not yet been validated to predict responses to ICI treatment in a large sample of cancer patients.

### DISCLOSURE OF THE INVENTION

### Problems to be Solved by the Invention

The present invention is to provide biomarker-searching devices and method that can predict effectiveness and overall survival of ICI treatment for cancer patients using network-based machine learning techniques.

The problems to be solved by the present application are not limited thereto, and should be interpreted as including all problems within the scope understood by those skilled in the art.

### Means for Solving the Problems

To solve the above-described technical problems, an aspect of the present disclosure provides a device for determining whether an immuno-oncology drug is effective to a cancer patient by a computing device, including a reactome pathway extraction unit configured to extract a target reactome pathway (functionally associated with a target) including the target of the immuno-oncology drug from a genomic network, a gene activity information conversion unit configured to convert gene activity information from transcriptome data of a target cancer patient, who will undergo the cancer immunotherapy, into activity information of the target reactome pathway, and a determination unit configured to determine whether the target cancer patient responds to the immuno-oncology drug by inputting target gene information into a pre-trained immuno-oncology drug response determination model.

Another aspect of the present disclosure provides a method for determining whether an immuno-oncology drug is effective to a cancer patient by a computing device, including a process of extracting a target reactome pathway including a target of the immuno-oncology drug from a genomic network, a process of converting gene activity information from transcriptome data of a target cancer patient, who will undergo cancer immunotherapy with the immuno-oncology drug, into activity information of the target reactome pathway, and a process of determining whether the target cancer patient responds to the immuno-oncology drug by inputting target gene information into a pre-trained immuno-oncology drug response determination model.

The means for solving the problems of the present disclosure are not limited to the above-described aspects and should be construed as including all means within a range that can be understood by a person with ordinary skill in the art.

### Effects of the Invention

When the device and the method according to the present disclosure are used, it is possible to detect a biomarker capable of accurately predicting effectiveness of ICI treatment on cancer patients and overall survival of the cancer patients. Accordingly, it is possible to maximize the effectiveness of ICI treatment.

### BRIEF DESCRIPTION OF THE DRAWINGS

FIG. 1 is a schematic diagram of a device according to the present disclosure.

FIG. 2 is a diagram illustrating the overall process of an algorithm according to the present disclosure.

FIG. 3A shows the prediction performance in terms of scores when NetBio-based prediction is combined with synthetic lethality-based prediction (SELECT score).

FIG. 3B shows the prediction performance in terms of scores when NetBio-based prediction is combined with synthetic lethality-based prediction (SELECT score).

FIG. 4A is a diagram illustrating a process of searching biomarkers associated with immunotherapy using network-based machine learning.

FIG. 4B is a diagram illustrating a process of searching biomarkers associated with immunotherapy using network-based machine learning.

FIG. 4C is a diagram illustrating a process of searching biomarkers associated with immunotherapy using network-based machine learning.

FIG. 5A is a diagram illustrating the performance of predicting drug responses and overall survival of immunotherapy-treated patients in four cohorts.

FIG. 5B is a diagram illustrating the performance of predicting drug responses and overall survival of immunotherapy-treated patients in four cohorts.

FIG. 5C is a diagram illustrating the performance of predicting drug responses and overall survival of immunotherapy-treated patients in four cohorts.

FIG. 5D is a diagram illustrating the performance of predicting drug responses and overall survival of immunotherapy-treated patients in four cohorts.

FIG. 6A is a diagram illustrating the prediction performance on a small-scale training sample by conducting a Monte Carlo cross-validation.

FIG. 6B is a diagram illustrating the prediction performance on a small-scale training sample by conducting a Monte Carlo cross-validation.

FIG. 6C is a diagram illustrating the prediction performance on a small-scale training sample by conducting a Monte Carlo cross-validation.

FIG. 7 is a diagram illustrating the prediction performance on three melanoma datasets.

FIGS. 8A-8E are diagrams illustrating the immunotherapy response prediction performance on external melanoma datasets which have not been used for training.

FIG. 9A shows the results of 22 prediction performance assessment tests using 8 different biomarkers.

FIG. 9B shows the results of 22 prediction performance assessment tests using 8 different biomarkers.

FIG. 9C shows the results of 22 prediction performance assessment tests using 8 different biomarkers.

FIGS. 10A-10C show the prediction performance when a genomic network is used (NetBio) or not used (ML-based feature selection).

FIGS. 11A-11D show the result of analyzing immunological characteristics of tumor microenvironment by NetBio-based prediction.

FIG. 12 shows the correlation between NetBio-based prediction and immunogenic features in a TCGA cohort.

FIG. 13A shows top 10 greatest immunogenic feature importance with positive coefficient.

FIG. 13B is a partial enlarged view of FIG. 13A.

FIG. 13C is a partial enlarged view of FIG. 13A.

FIG. 14A shows top 10 greatest immunogenic feature importance with negative coefficient.

FIG. 14B is a partial enlarged view of FIG. 13A.

FIG. 14C is a partial enlarged view of FIG. 13A.

FIG. 15 shows that the expression levels of NetBio pathways (mitotic G2-G2-M phases) are in positive correlation with follicular helper T cell proportions in TCGA gastric cancer.

FIGS. 16A-16B show that the expression levels of NetBio pathways (“chemokine receptors bind chemokines” and “FcgR activation”) are in positive correlation with leukocyte fractions.

FIGS. 17A-17C show that the expression levels of NetBio pathways are consistent with immunohistochemistry-based immune phenotypes in bladder cancer.

FIGS. 18A-18F show that the prediction performance of the overall survival in PD-L1 inhibitor (atezolizumab)-treated patients when network-based transcriptome features are combined with the tumor mutation burden (TMB).

FIGS. 19A-19C shows TMB-based PD-L1 response predictions and compares TMB-based predictions with NetBio-based predictions.

FIG. 20 shows the TMB levels of predicted ICI responders and non-responders in IMvigor210 datasets.

FIG. 21 is a flowchart showing an algorithm according to the present disclosure.

### BEST MODE FOR CARRYING OUT THE INVENTION

Hereafter, examples of the present disclosure will be described in detail with reference to the accompanying drawings so that the present disclosure may be readily implemented by a person with ordinary skill in the art. However, it is to be noted that the present disclosure is not limited to the examples but can be embodied in various other ways. In the drawings, parts irrelevant to the description are omitted for the simplicity of explanation, and like reference numerals denote like parts through the whole document.

Throughout this document, the term “connected to” may be used to designate a connection or coupling of one element to another element and includes both an element being “directly connected to” another element and an element being “electronically connected to” another element via another element.

Further, through the whole document, the term “comprises or includes” and/or “comprising or including” used in the document means that one or more other components, steps, operation and/or existence or addition of elements are not excluded in addition to the described components, steps, operation and/or elements unless context dictates otherwise.

Through the whole document, the term “about or approximately” or “substantially” is intended to have meanings close to numerical values or ranges specified with an allowable error and intended to prevent accurate or absolute numerical values disclosed for understanding of the present disclosure from being illegally or unfairly used by any unconscionable third party. Through the whole document, the term “step of” does not mean “step for”.

Through the whole document, the term “combination(s) of” included in Markush type description means mixture or combination of one or more components, steps, operations and/or elements selected from a group consisting of components, steps, operation and/or elements described in Markush type and thereby means that the disclosure includes one or more components, steps, operations and/or elements selected from the Markush group.

Through the whole document, a phrase in the form “A and/or B” means “A or B, or A and B”.

Through the whole document, the term “genomic network” refers to various gene interactions among in vivo genes. For example, the genomic network may be a Protein-Protein Interaction network.

The gene interactions include physical proximity on a chromosome, coexistence in an evolution process, similarity in expression level, physical association of expressed proteins, locus heterogeneity in phenotype such as disease, etc. A gene determines morphological and physiological characteristics of a subject and thus is greatly relevant to health conditions of a living organism. Therefore, research on the gene interactions is significant in that it is possible to identify the comprehensive role of a plurality of genes with respect to phenotypes of a subject, such as diseases or responses to drugs.

The present invention relates to network-based machine learning framework that can (i) make robust predictions across ICI datasets and (ii) identify novel potential biomarkers. Specifically, the present invention could robustly predict responders and non-responders using the expression levels of network-based biomarkers in more than 700 patient samples, covering melanoma, metastatic gastric and bladder cancer patients treated with ICIs targeting the PD1/PD-L1 axis. To identify robust drug response biomarkers, a network-based approach was implemented, in which biological pathways located proximal to immunotherapy targets in a PPI network were identified.

A first aspect of the present disclosure provides a device for determining whether an immuno-oncology drug is effective to a cancer patient by a computing device, including a reactome pathway extraction unit configured to extract a target reactome pathway including a target of the immuno-oncology drug from a genomic network, a gene activity information conversion unit configured to extract target gene information corresponding to the target reactome pathway from transcriptome data of a target cancer patient who will undergo cancer immunotherapy with the immuno-oncology drug, and a determination unit configured to determine whether the target cancer patient responds to the immuno-oncology drug by inputting the target gene information into a pre-trained immuno-oncology drug response determination model.

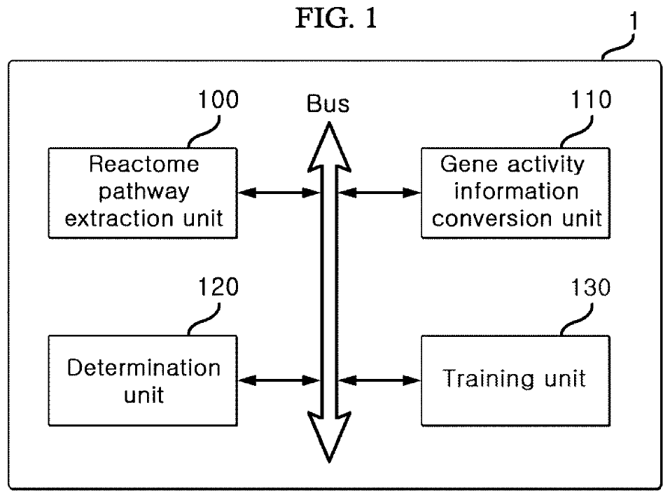

The pathway extraction unit may be configured to prepare the genomic network and search network-based biomarkers (see FIG. 1).

### Preparation of Genomic Network

The human PPI network was downloaded (https://string-db.org/) from the STRING database v.11.0. To leverage high-confidence PPIs, links with interaction scores greater than 700 were considered. Next, for network-based analysis in this manuscript, the largest connected component of the PPI network was used, resulting in 16,957 nodes and 420,381 edges. The largest connected component was computed using the NetworkX python module. Cytoscape (v.3.7.1) was used for network visualization.

### Network-Based Biomarker (NetBio) Detection

The detection of NetBio pathways comprises two steps: (i) the detection of ICI target-proximal genes in the PPI network and (ii) detection of biological pathways (Reactome pathway) proximal to ICI targets (i.e., NetBio pathways). First, ICI target-proximal genes were identified via network propagation using the page-rank algorithm from the NetworkX python module. One for ICI targets and zero for all other genes in the network as an input for the personalization parameter in the page-rank algorithm were used. Default settings were used for any other parameters for the page-rank algorithm. After network propagation, the top 200 genes with highest influence scores were considered as ICI target-proximal genes.

Next, biological pathways located proximal to ICI targets were detected using ICI target-proximal genes. The gene set enrichment test that specifically calculates how many ICI target-proximal genes are included in each pathway, was computed. The hypergeometric test was used to obtain statistical significance. Finally, pathways significantly enriched with ICI target-proximal genes were selected using an adjusted P-value of less than 0.01. Hypergeometric test statistics and the adjusted P-value were computed using scipy and statsmodels python modules, respectively.

The gene activity information conversion unit may be configured to process patient data.

### Curation and Preprocessing of Patient Data

The data of the following 7 different patient cohorts treated with ICIs targeting the PD-1/PD-L1 axis were collected:
(1) Gide et al. (Nivolumab, Pembrolizumab and/or Ipilimumab treated melanoma, n=91; Gide, T. N. et al. Distinct Immune Cell Populations Define Response to Anti-PD-1 Monotherapy and Anti-PD-1/Anti-CTLA-4 Combined Therapy. Cancer Cell 35, 238-255.e6 (2019))
(2) Liu et al. (Nivolumab or Pembrolizumab treated melanoma, n=121; Liu, D. et al. Integrative molecular and clinical modeling of clinical outcomes to PD1 blockade in patients with metastatic melanoma. Nat. Med. 25, 1916-1927 (2019).)
(3) Kim et al. (Pembrolizumab treated metastatic gastric cancer, n=45; Kim, S. T. et al. Comprehensive molecular characterization of clinical responses to PD-1 inhibition in metastatic gastric cancer. Nat. Med. 24, 1449-1458 (2018).)
(4) IMvigor210 (Atezolizumab treated bladder cancer, n=348; Mariathasan, S. et al. TGF ß attenuates tumour response to PD-L1 blockade by contributing to exclusion of T cells. Nature (2018). doi: 10.1038/nature25501)
(5) Auslander et al. (anti-PD-1 and/or anti-CTLA4 treated melanoma, n=37; Auslander, N. et al. Robust prediction of response to immune checkpoint blockade therapy in metastatic melanoma. Nat. Med. (2018). doi: 10.1038/s41591-018-0157-9)
(6) Prat et al. (Nivolumab or Pembrolizumab treated melanoma, n=25; Prat, A. et al. Immune-Related Gene Expression Profiling After PD-1 Blockade in Non-Small Cell Lung Carcinoma, Head and Neck Squamous Cell Carcinoma, and Melanoma. Cancer Res. 77, 3540-3550 (2017).)
(7) Riaz et al. (Nivolumab treated melanoma, n=49; Riaz, N. et al. Tumor and Microenvironment Evolution during Immunotherapy with Nivolumab. Cell 171, 934-949.e16 (2017).)

For the dataset (6), only melanoma samples were considered. For the dataset (7), only expression samples collected before drug treatment was used.

The information of each cohort is as Table 1 below.

TABLE 1

| | | | Treatment | Total | | |
|---|---|---|---|---|---|---|
| Dataset | Cancer type | Drug | type | samples | Responders | Nonresponders |
| Mariathasan | Bladder | Atezolizumab | Pre | 348 | 60 | 238 |
| (IMvigor210) | | (PD-L1) | | | | |
| Liu | Melanoma | anti PD1 | Pre | 121 | 49 | 72 |
| | | (Nivolumab, | | | | |
| | | Pembrolizumab) | | | | |
| Riaz_pre | Melanoma | Nivolumab | Pre | 49 | 10 | 39 |
| | | (PD1) | | | | |
| Gide | Melanoma | Ipilimumab (CTLA4), | Pre | 91 | 49 | 42 |
| | | Nivolumab (PD1) | | | | |
| | | Pambrolizumab (PD1) | | | | |
| Kim | Metastatic | anti PD1 | Pre | 45 | 12 | 33 |
| | gastric | (Nivolumab, | | | | |
| | cancer | Pembrolizumab) | | | | |
| Auslander | me | ICB | Pre/On | 37 | 3 | 34 |
| Prat_MELANOMA | me | ICB | Pre | 25 | 9 | 18 |

Pre: pre-treatment; means that sample was collected before drug treatment.

On: on-treatment; means that sample was collected after drug treatment Regarding the TCGA dataset, the following were used: (1) TCGA SKCM (melanoma, n=103), (2) TCGA STAD (stomach adenocarinoma, n=375) and (3) TCGA BLCA (bladder cancer, n=405). Gene expression data (HTSeq-Counts), somatic mutation data and clinical data (i.e. overall survival data) were downloaded using the TCGAbiolinks R package. To calculate the TMB in TCGA cancer patients, following equation from Wang et al. was used (Wang, X. & Li, M. Correlate tumor mutation burden with immune signatures in human cancers. BMC Immunol. (2019). doi: 10.1186/s12865-018-0285-5)

$${TMB}_{patient} = {T}_{patient} \times 2. + {NT}_{patient} \times 1.$$

wherein,
Tₚₐₜᵢₑₙₜ is total number of truncating mutations.
NTₚₐₜᵢₑₙₜ is the total number of non-truncating mutations.

For truncating mutations, nonsense mutations were considered, frame-shift deletion or insertion and splice-site mutations. For non-truncating mutations, missense mutations were used, in-frame deletion or insertion, and nonstop mutations.

For the pre-processing of gene expression data, the gene expression levels using read counts were calculated from the IMvigor210, Auslander, Prat, Riaz and TCGA datasets, which were normalized using trimmed means of M-values normalization from the edgeR R package. For other datasets, normalized expression values provided by Lee et al. (https://zenodo.org/record/4661265) were used. To estimate the pathway expression levels, Reactome pathways downloaded from the MSigDB database (Lee, J. S. et al. Synthetic lethality-mediated precision oncology via the tumor transcriptome. Cell (2021). doi:10.1016/j.cell.2021.03.030) were used, and performed single-sample GSEA (ssGSEA) using the GSVA R package (Hanzelmann, S., Castelo, R. & Guinney, J. GSVA: Gene set variation analysis for microarray and RNA-Seq data. BMC Bioinformatics (2013). doi: 10.1186/1471-2105-14-7) The normalized enrichment score (NES) was used to estimate the pathway expression levels of each sample.

To classify samples into responders and non-responders, response evaluation criteria was used in solid tumors (RECIST) criteria, where complete response (CR) and partial response (PR) were classified as responders and stable disease (SD) and progressive disease (PD) were classified as non-responders. For datasets that did not provide or use RECIST criteria, responder and non-responder classification were used from the datasets.

The determination unit may be configured to measure the performance of machine learning predictions and the performance of prediction for a combined model using NetBio-based predictions and SELECT (synthetic lethal relation)-based predictions.

### Measuring Performances of Machine-Learning (ML) Predictions

Logistic regression was used to train ML models, implemented in Scikit-learn in Python. Specifically, the 12 regularized logistic regression model was used. To train ML models, the expression levels of genes/pathways against drug responses (classified as responders and nonresponders) were used. To select optimal hyper-parameters, five-fold cross-validation was conducted in a training dataset by iterating the regularization parameter (C) from 0.1 to 1 in 0.1 intervals. “balanced” parameters were used for class weight hyper-parameters to reduce class imbalance effects. To identify optimal hyper-parameters, the GridSearchCV function from the Scikit-learn module was used. The gene/pathway expression levels are z-score-standardized before ML training/testing to minimize the batch effect between cohorts.

For LOOCV (Leave-one-out cross validation), cohorts that agree with the following criteria were considered: (i) cohorts with more than 30 samples and (ii) at least 10 samples for both responders and non-responders. Four datasets remained after applying the criteria (Gide, Liu, Kim and Imvigor210). The LeaveOneOut function from the Scikit-learn module was used to split the training and test datasets.

For predictions based on genes (GeneBio) and the tumor microenvironment (TME-Bio), gene expression levels were used to train/test the ML model. For GeneBio, the expression levels of PD-1, PD-L1 or CTLA4 were used. For TME-Bio, the gene expression levels of markers of (i) CD8 T cells, (ii) T cell exhaustion, (iii) cancer-associated fibroblasts and (iv) tumor-associated macrophages (M2 macrophages) were used.

To test the performance of data-driven ML predictions, feature selection was conducted using the SelectKBest function from Scikit-learn (‘f_classif’ was used for the score function parameter). K number of reactome pathways was selected, where K equals the number of NetBio pathways. To train and test the data-driven ML model, the pathway expression levels were.

Calculating Prediction Performances for the Combined Model Using NetBio-Based Predictions and Predictions from Synthetic Lethal Relationship (SELECT)

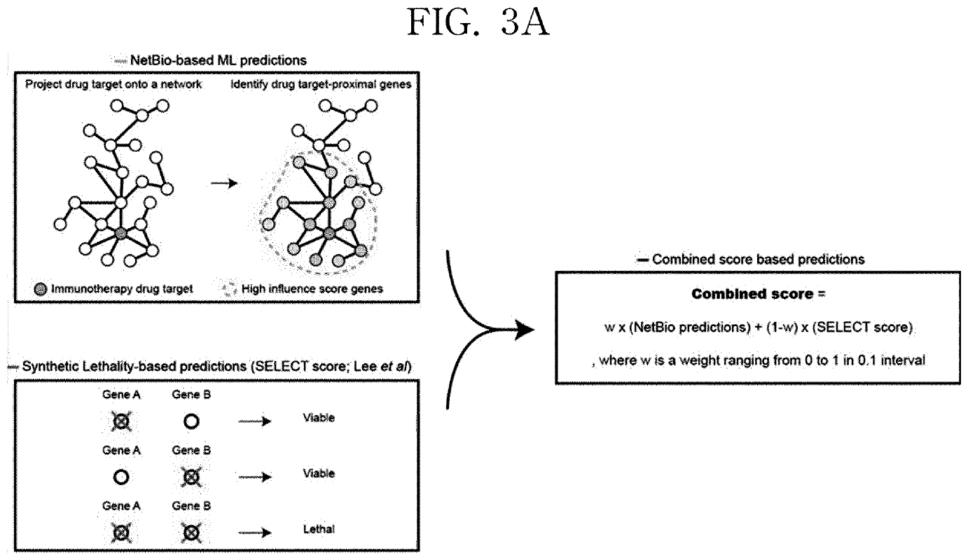

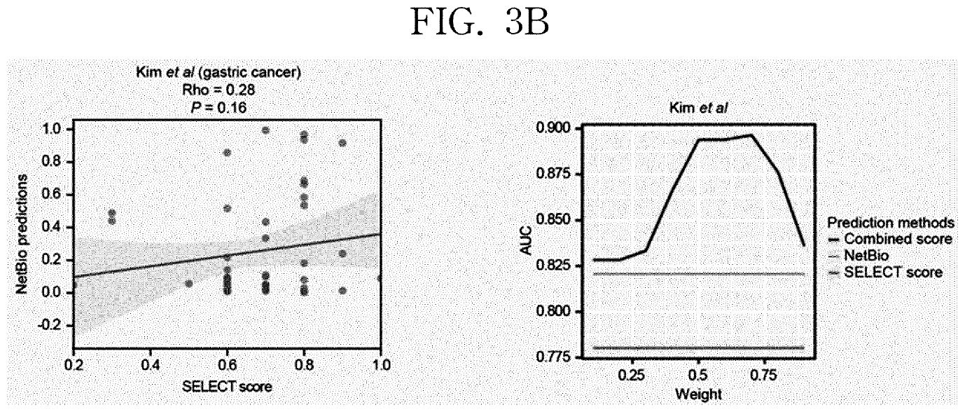

The SELECT score was provided by the original authors by personal communication. SELECT uses synthetic lethal and synthetic rescue relationships between two genes identified from non-ICI-treated cancer samples. Before combining the SELECT score with NetBio-based predictions (using the prediction probability from LOOCV), first Spearman's correlation between the two prediction scores was computed. In the Kim et al cohort (metastatic gastric cancer), the two prediction scores showed no correlation with each other (Spearman's correlation rho=0.28; P=0.16; FIG. 3B), suggesting that the two different prediction models captured distinct biological signals.

To combine the SELECT score with NetBio-based predictions, the linear weighted model by Zhang et al. was used (Zhang, N. et al. Predicting Anticancer Drug Responses Using a Dual-Layer Integrated Cell Line-Drug Network Model. PLOS Comput. Biol. (2015). doi:10.1371/journal.pcbi.1004498):

Combined score=w×(NetBio-based predictions)+(1−w)×(SELECT score)
wherein, w is the linear weight ranging from 0 to 1 in 0.1 intervals.

The AUC of the receiver operating characteristics curve was used as a performance metric.

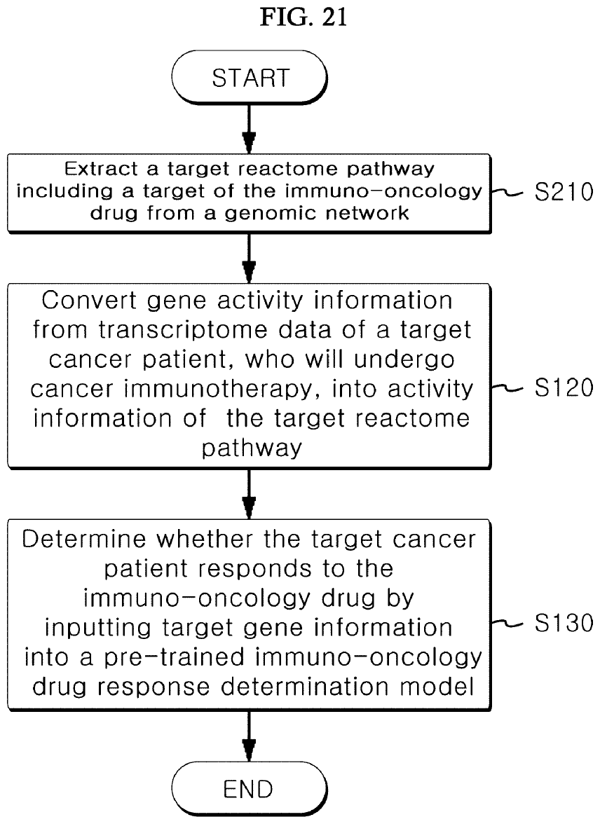

A second aspect of the present disclosure provides a method for determining whether an immuno-oncology drug is effective to a cancer patient by a computing device, including a process of extracting a target reactome pathway including a target of the immuno-oncology drug from a genomic network, a process of extracting target gene information corresponding to the target reactome pathway from transcriptome data of a target cancer patient who will undergo cancer immunotherapy with the immuno-oncology drug, and a process of determining whether the target cancer patient responds to the immuno-oncology drug by inputting the target gene information into a pre-trained immuno-oncology drug response determination model (see FIG. 21).

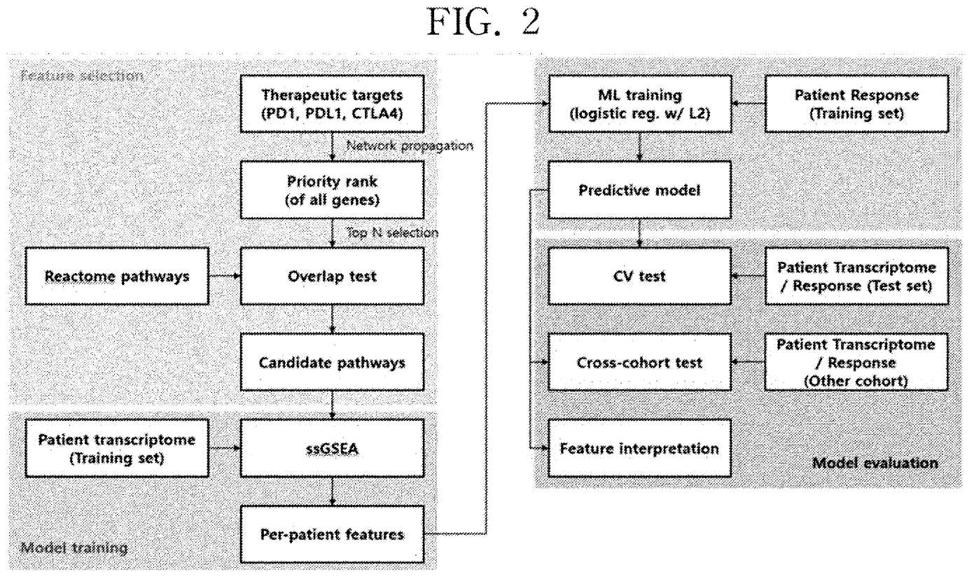

The features described above in respect of the first aspect of the present disclosure may equally apply to the second aspect of the present disclosure. The overall process of an algorithm is illustrated in FIG. 2.

Hereinafter, embodiments and examples of the present disclosure will be described in detail with reference to the accompanying drawings. However, the present disclosure may not be limited to the following embodiments, examples, and drawings.

### Example 1. Data Pre-Processing and Training of Machine Learning Model

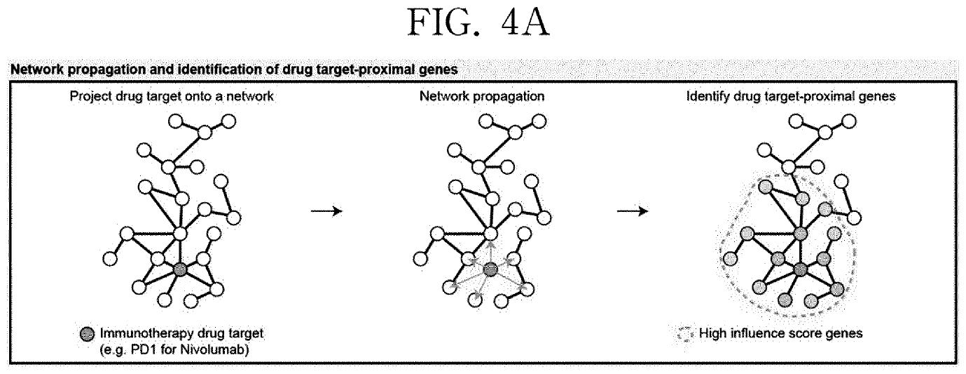

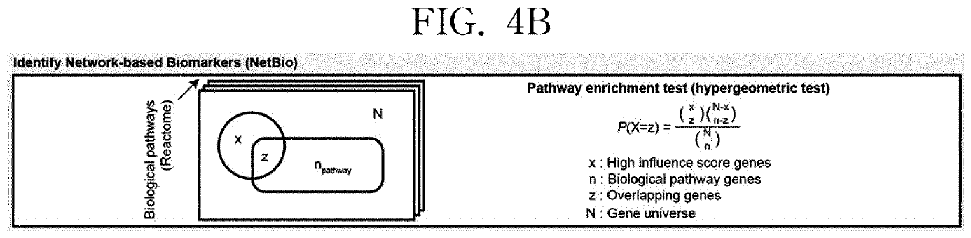

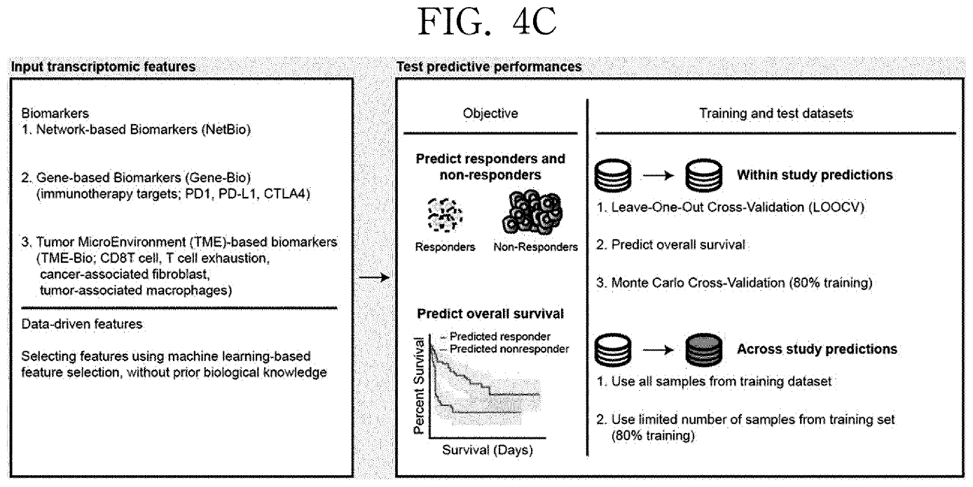

The STRING PPI network, comprising 16,957 nodes and 420,381 edges was used. First, network propagation was applied, using ICI targets (e.g., PD1 for nivolumab or PD-L1 for atezolizumab) as seed genes, to spread the influence of ICI targets over the network (FIG. 4A). Next, genes with high influence scores (top 200 genes) were selected and identified biological pathways (Reactome pathways) enriched with the genes (FIG. 4B). Then the selected biological pathways were used to predict the immunotherapy response and considered these pathways as Network-Based Biomarkers (NetBio).

To conduct ML-based immunotherapy response predictions, NetBio was used as input features; as a negative control, gene-based biomarkers were used (i.e., immunotherapy target genes; GeneBio), tumor microenvironment-based biomarkers (TME-Bio) or pathways selected from data-driven ML approaches (FIG. 4C). Using the expression levels of the input features, logistic regression was applied to train the ML model. To test the predictive performances of the input features, the performance was measured in predicting (i) the drug response measured by a reduced tumor size after immunotherapy treatment or (ii) the patient's overall survival. To train an ML model using supervised learning, different combinations of training and test datasets were used to extensively measure the consistency of the prediction performances. Specifically, (i) within-study predictions, in which training, and test datasets were generated from a single cohort or (ii) across-study predictions, in which two independent datasets were used as training and test datasets were performed. Furthermore, using large or small numbers of training samples was alternated to measure the consistency of the prediction performances under various training conditions.

### Example 2. Prediction Performance by NetBio-Based Machinelearning Through within-Study Cross-Validation

The transcriptome of NetBio could make consistent predictive performances to predict the ICI response. In comparison, less stronger prediction performances was observed when using the expression of drug targets (i.e., PD-1 for nivolumab and pembrolizumab, PD-L1 for atezolizumab and CTLA4 for ipilimumab-treated patients).

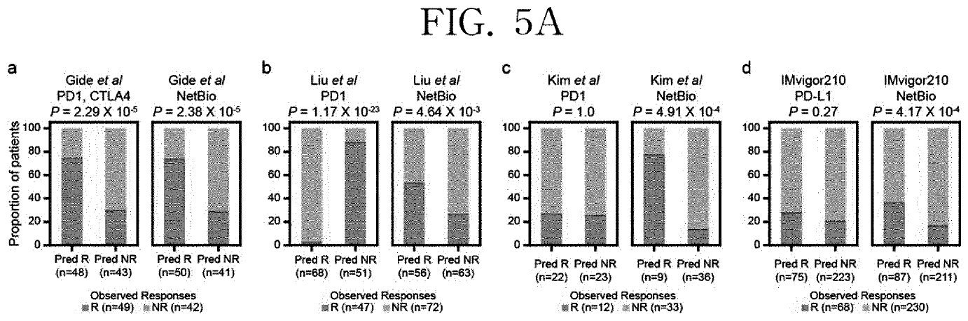

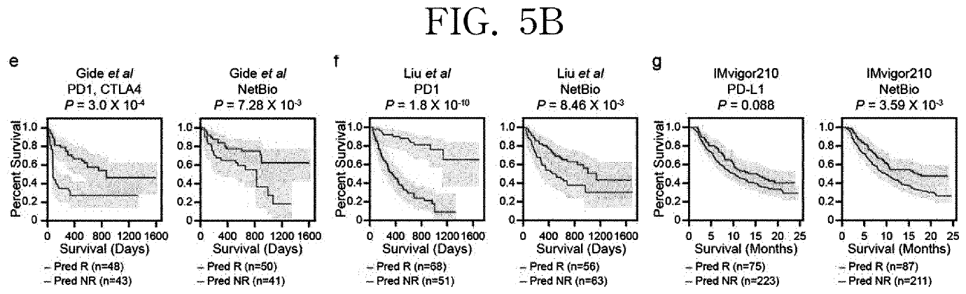

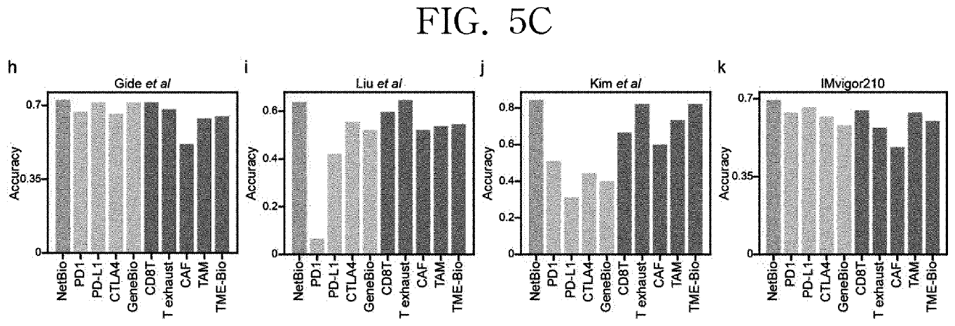

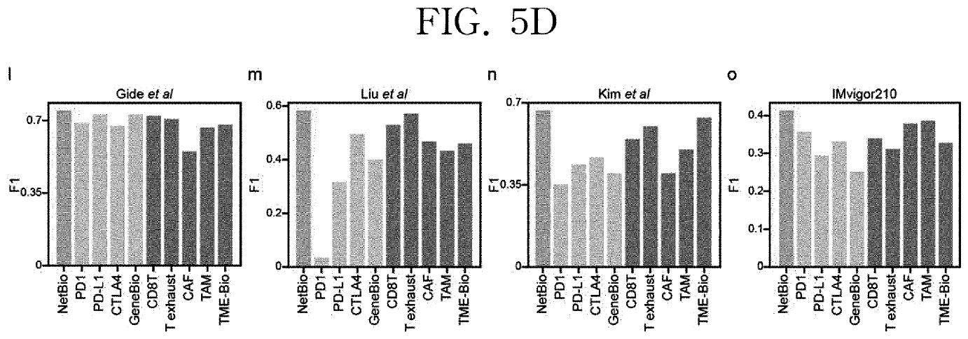

First a leave-one-out cross-validation (LOOCV) was conducted to measure the performance using NetBio or other known immunotherapy-related biomarkers (including drug targets). To this end, four immunotherapy cohorts were used-two melanoma cohorts (Gide et al., Liu et al.), one metastatic gastric cancer cohort (Kim et al.) and one bladder cancer cohort (IMvigor210). The ML model trained using the NetBio consistently made accurate predictions in all four datasets (a-d of FIG. 5A; Fisher's exact test, P<0.05 was considered significant). By contrast, predictions made using the expression levels of drug targets were less consistent, where drug targets were accurately predictive only in a melanoma cohort (Gide et al.) but not in the other three cancer cohorts. Notably, predictions using the expression level of drug targets were inversely predictive in the Liu dataset.

Furthermore, a prolonged overall survival was consistently observed for patients predicted as ICI responders using the NetBio-based ML in three datasets with overall survival data available (Gide, Kim and Imvigor210; log-rank test P<0.05 was considered significant); using drug target expression predicted the overall survival in only one dataset (e-g of FIG. 5B). Altogether, the data showed that the network-based approach, which expands biomarkers to network neighbors of drug targets, improves predictions based on the expression levels of drug targets.

Next the predictive performance of the NetBio with other previously identified ICI-related biomarkers, including GeneBio or TME-Bio, were compared and it was found that approach of the present application was, in most cases, better across all four cancer datasets. For GeneBio, the expression levels of immunotherapy targets (PD-1, PD-L1, or CTLA4) were considered; for TME-Bio, gene sets associated with CD8 T cell proportions, T cell exhaustion, CAFs (cancer associated fibroblasts), and TAMs (tumor associated macrophages) were considered. Accuracy and the FI score were used to measure the predictive performances of LOOCV and found that NetBio-based predictions were better in 55 of 56 comparisons (98.2%) than predictions using all other biomarkers (FIG. 5C, FIG. 5D).

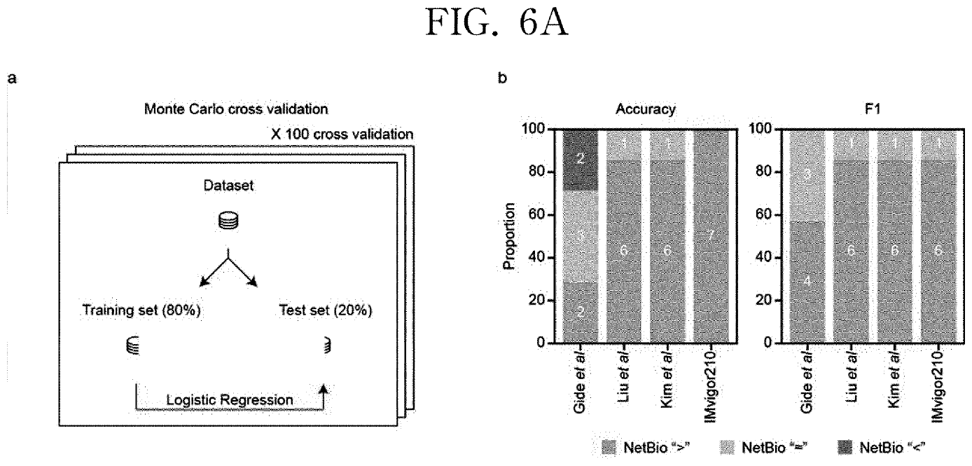

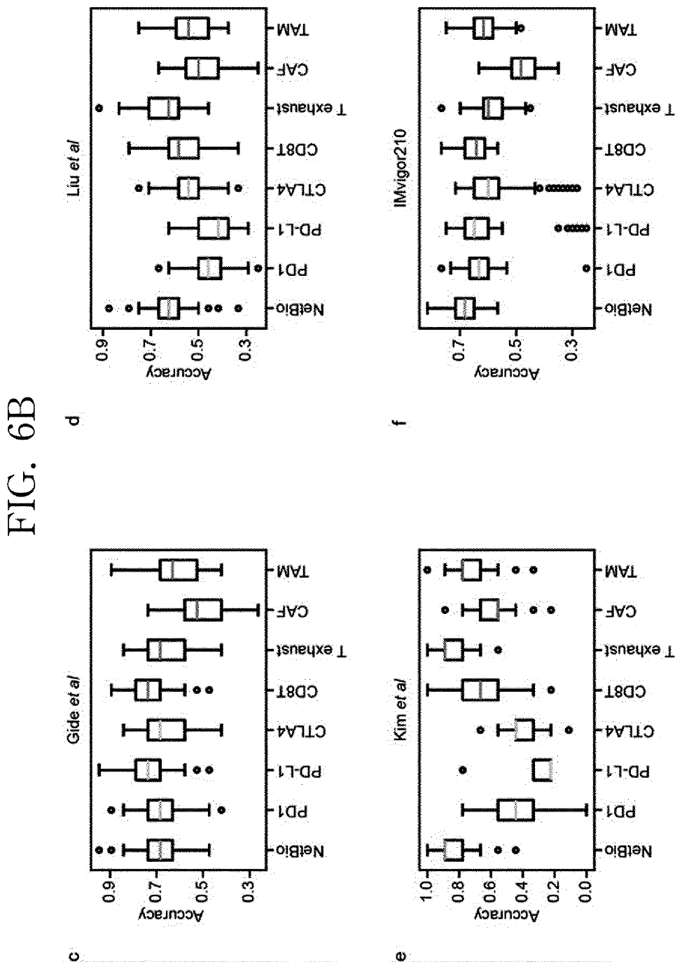

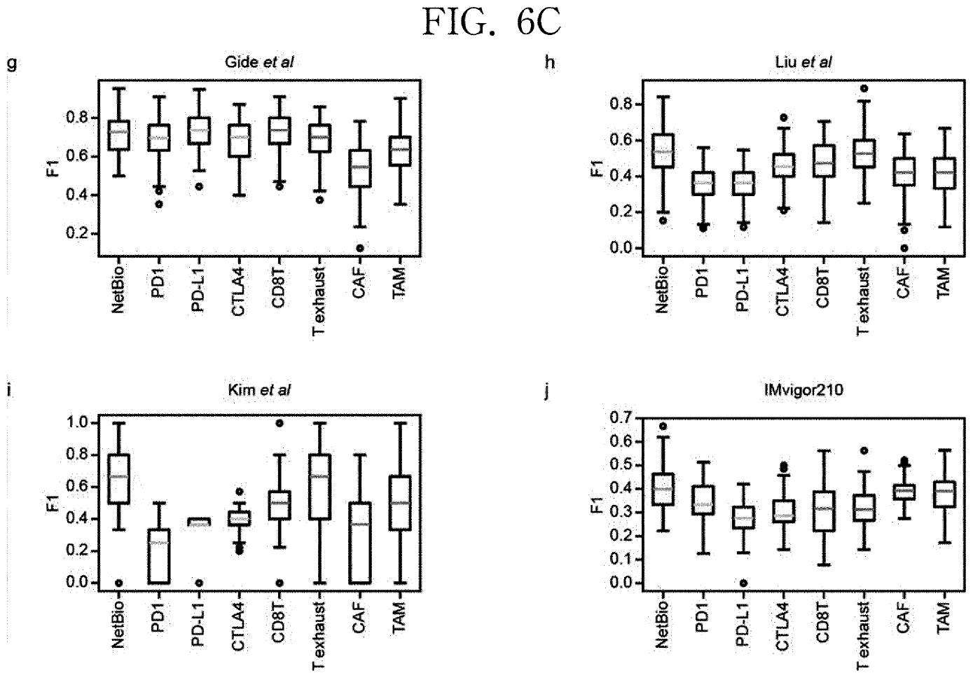

Furthermore, predictions from NetBio were similar to or better than other biomarkers when using fewer training datasets to train ML models. Specifically, a Monte Carlo cross-validation was conducted. For 100 different iterations, 80% of the samples were randomly selected and used as a training set and the remaining 20% were used as a test set (a of FIG. 6A). In 54 of 56 comparisons (96.4%), the network-based approach showed significantly better or equal performance compared with GeneBio or TME-Bio (b of FIG. 6A, FIG. 6B, FIG. 6C; two-sided Student t-test P<0.05 was considered significant). The results provide further evidence that using a network-based approach to identify biomarkers can make robust predictions of the ICI response in cancer patients.

### Example 3. Prediction Performance of Netbio-ML in Additional Independent Melanoma Datasets

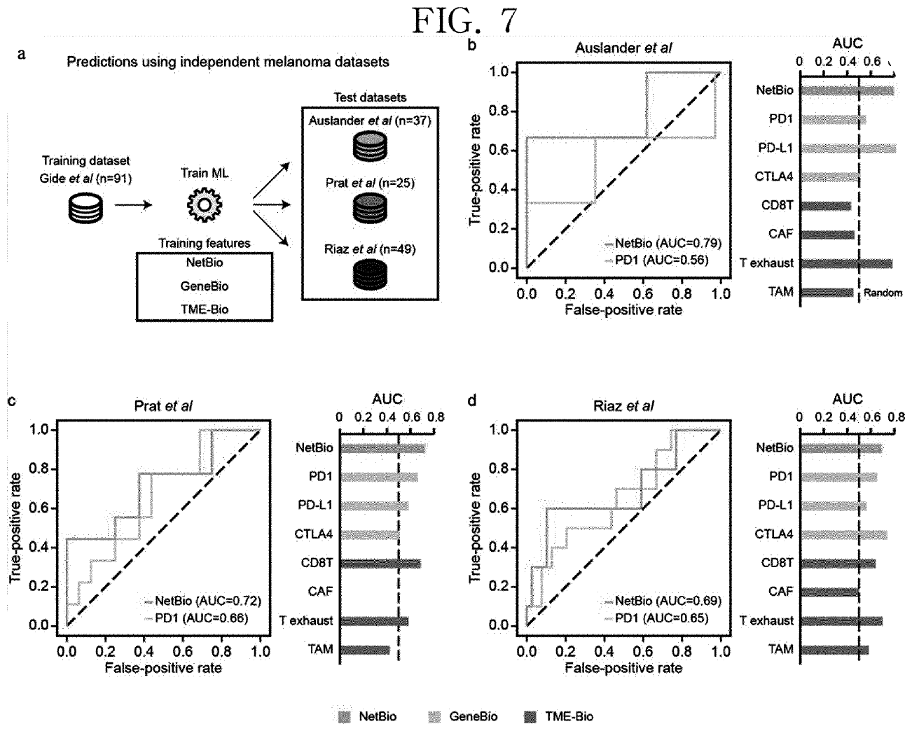

Key aspects of an accurate ML model include the following: (i) its ability to generalize to new datasets and (ii) its consistent performance when few training samples are available. First, it is observed that the ML model trained using NetBio could make robust predictions when using independent datasets, whereas GeneBio or TME-Bio was less predictive of the drug response (FIG. 7). To test the generalizability of the ML model, the melanoma dataset from Gide et al was used to train the ML model and tested the predictive performance in three independent melanoma datasets (a of FIG. 7; Auslander et al., Prat et al., and Riaz et al.). To compute the performance of the model, the prediction probability using a logistic regression model was used. The area under the curve (AUC) of the receiver operating characteristics curve was selected as a performance metric. NetBio-based ML showed AUCs greater than 0.7 in two external datasets (b, c of FIG. 7, Auslander AUC=0.79, Prat AUC=0.72), and 0.69 in the remaining dataset (d of FIG. 7; Riaz). In contrast to NetBio-based ML, predictions using GeneBio or TME-Bio displayed highly varying prediction performances (b-d of FIG. 7). For example, PD-1 expression showed fewer optimal performances, with the maximum AUC reaching only 0.66. Additionally, although predictions using markers of T cell exhaustion were highly accurate in the Auslander and Riaz datasets (AUC >0.7), the prediction performances were slightly better than random expectation in the Prat dataset (c of FIG. 7, AUC=0.58).

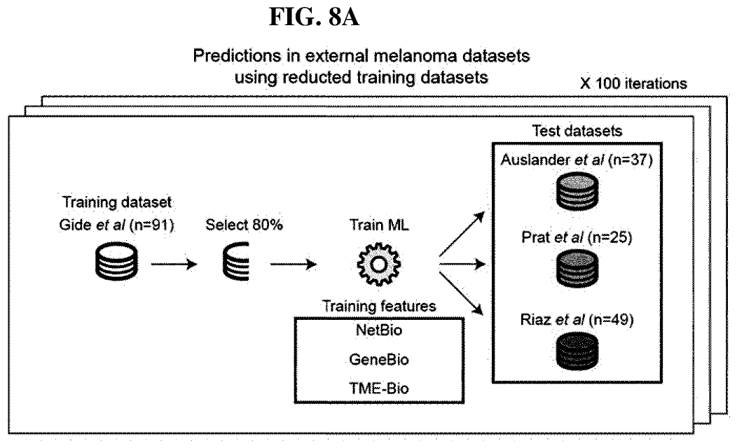

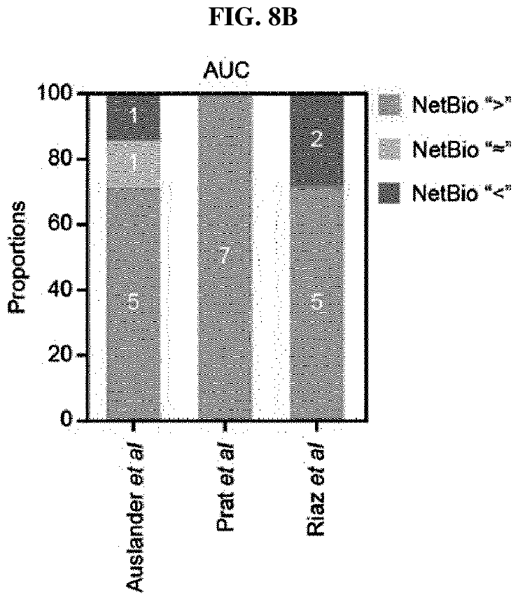

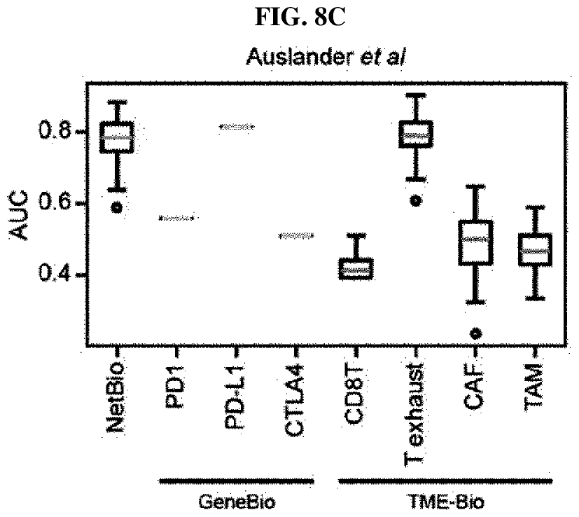

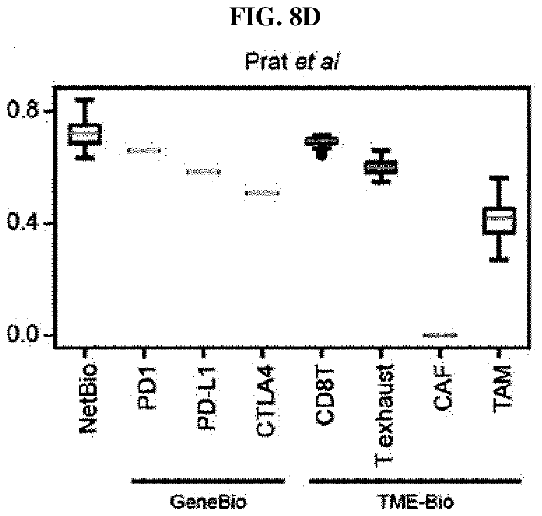

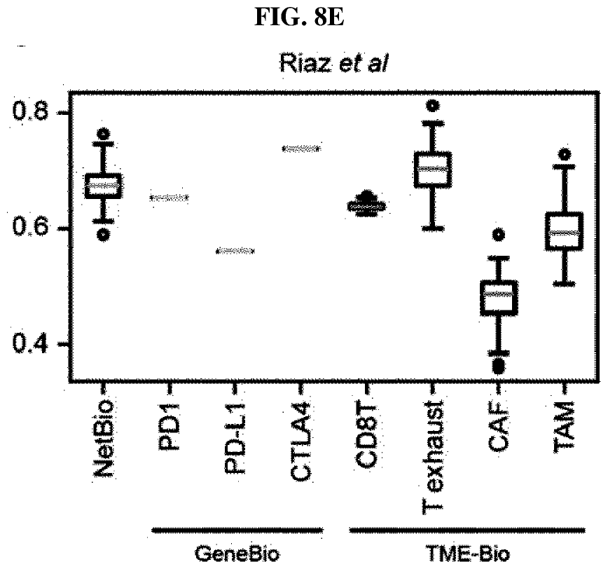

Next, whether the ML model can make robust predictions was tested even when fewer training samples are available. Again, NetBio-based ML with smaller sample sizes made consistent predictions compared with GeneBio or TME-Bio-based ML models. To test this, for 100 iterations, 80% of patients from the training dataset (Gide dataset) were randomly sampled to train the ML model and tested the prediction performance in three external melanoma datasets (a of FIG. 8). The biomarkers showed statistically significantly better or equal performance in 18 of 21 comparisons (b of FIG. 8; 85.7%) Only PD-L1 expression in the Auslander dataset, CTLA4 in the Riaz dataset and CD8 T cell exhaustion markers in the Riaz datasets displayed prediction performances that were better than NetBio-based predictions, but these biomarkers (PD-L1, CTLA4, and CD8 T exhaustion markers) were inconsistent in their predictions in the other melanoma datasets (b-e of FIG. 8).

### Example 4. Overall Comparison of Performance Based on NetBio, Bene Bio, and TME-Bio

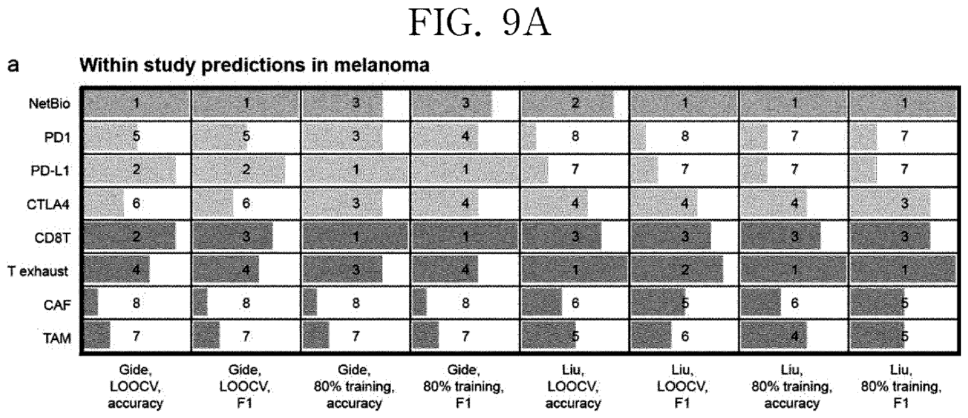

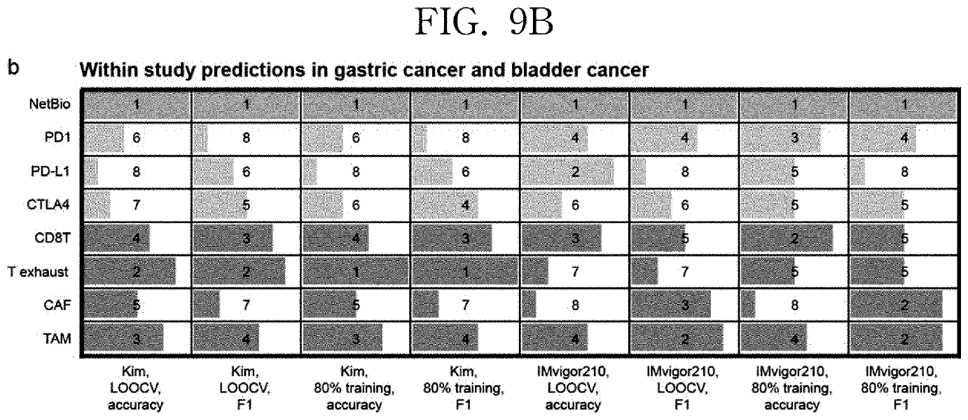

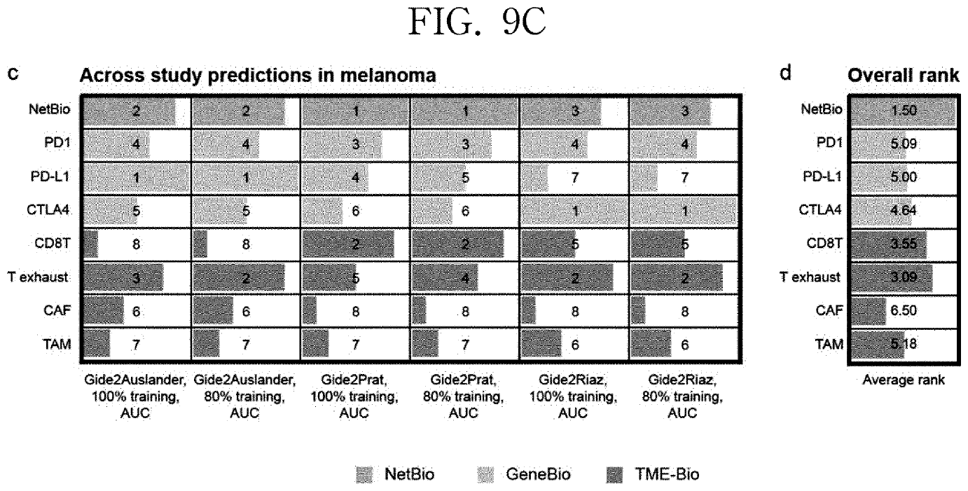

Overall, the NetBio-based ML model was robust in accurately predicting the ICI response in cancer patients (FIGS. 9A-9C). Among 22 different tests that have been conducted, NetBio showed equal or better performance in 143 of 154 comparisons (92.9%), with an overall average prediction rank of 1.5 among 8 different biomarkers (d of FIG. 9C). This finding suggests that NetBio enables improved predictions compared with GeneBio or TME-Bio based predictions. Markers of CD8 T cell exhaustion and CD8 T cells were the next best performers (average ranks of 3.09 and 3.55, respectively), an expected finding considering that ICIs aim to reinvigorate CD8 T cells to kill cancer cells. The existence of CD8 T cells in the vicinity of a tumor correlates with the ICI response, and naturally identifying hot or cold tumors are actively being investigated for their clinical usefulness. However, compared with predictions made using CD8 T cell markers or CD8 T cell exhaustion markers, NetBio showed equal or better performance in 20 of 22 tests (90.9%) or 19 of 22 tests (86.4%), respectively (FIGS. 9A-9C). Furthermore, in PD-L1 treated bladder cancer patients, although NetBio-based predictions consistently ranked first throughout four different prediction tasks, markers of CD8 T cell exhaustion were not good predictors of a response. These results suggest that (i) a distinct immune escape mechanism exists for different cancer types and (ii) NetBio-based predictions robustly predict immunotherapy responses.

### Example 5. Comparison Between NetBio-Based Predictions and Purely Data-Driven Feature Selection

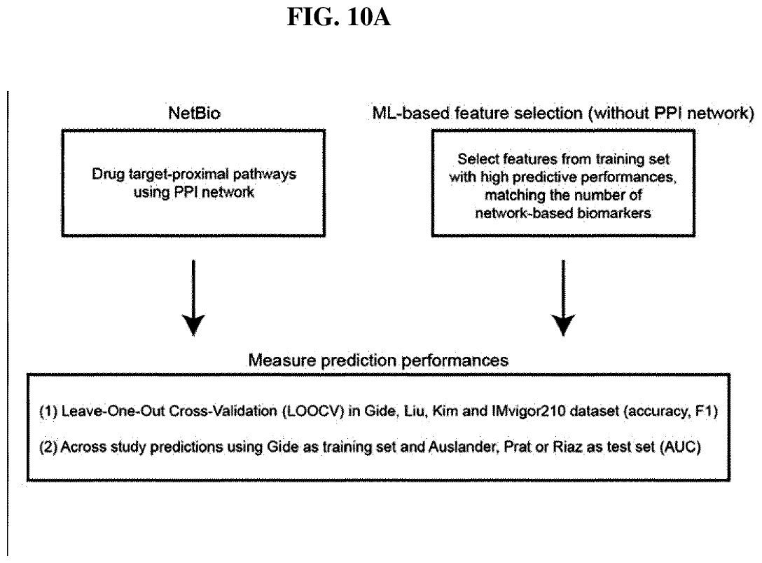

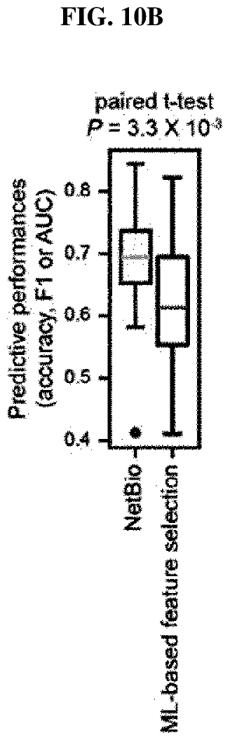

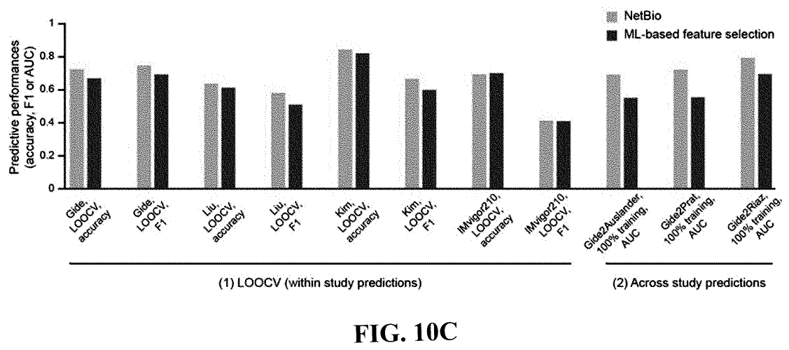

A major limitation of using data-driven ML models for clinical applications are its inability to consistently perform in new datasets, despite performing well in training datasets. Thus, whether the addition of prior biological knowledge, representing a PPI network in this study, can improve feature selection compared with purely data-driven feature selection approaches was tested. The NetBio-based ML model enables consistently improved prediction performances compared with purely data-driven ML predictions. In detail, for the data-driven ML model, K number features (where K equals the number of NetBio) that best distinguish responders and non-responders in a training dataset were selected and the selected features were used to train the ML model (a of FIG. 10). In 11 different tasks, it was found that NetBio-based predictions showed significantly better performance than features from ML-based feature selection (b of FIG. 10; two-sided paired Student t-test P-value=3.3×10⁻³). Furthermore, performance improvements were consistently observed when predicting across melanoma cohorts (c of FIG. 10), suggesting that network-guided selection can help reduce the overfitting of ML models. This observation suggests that network-guided feature selection can provide robust features compared with those from purely data-driven feature selection. Altogether, the result suggests that robust transcriptomic biomarkers can be identified by leveraging network-based biomarker selection.

### Example 6. Prediction Performance of NetBio-Based Prediction in External the Cancer Genome Atlas (TCGA) Datasets

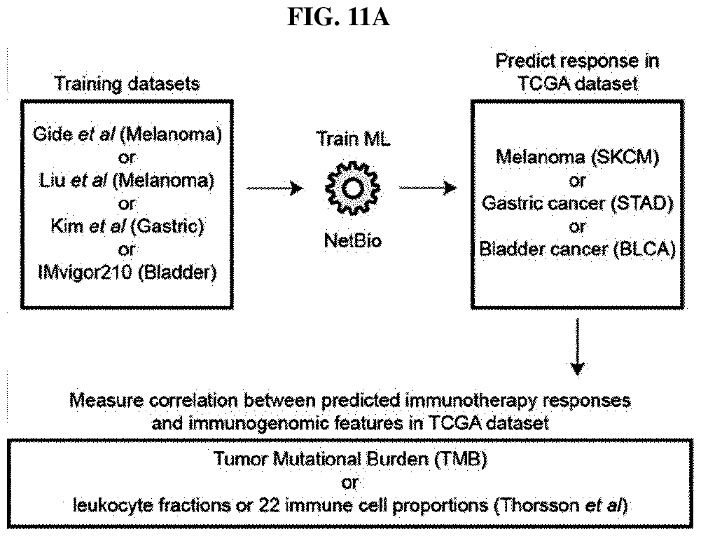

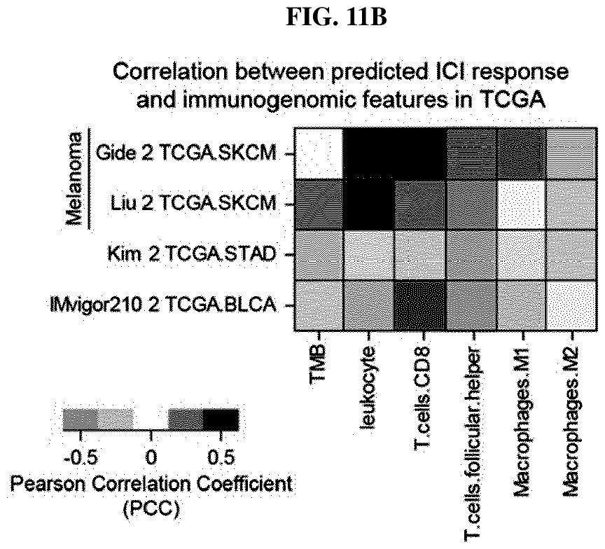

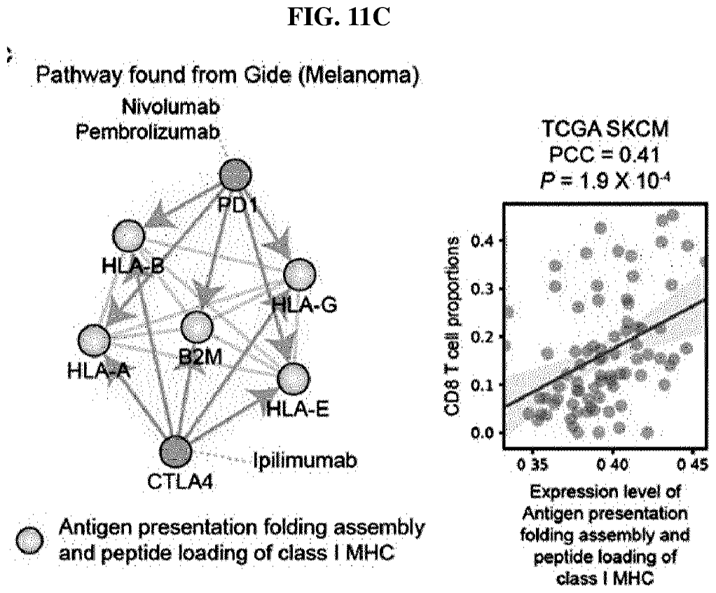

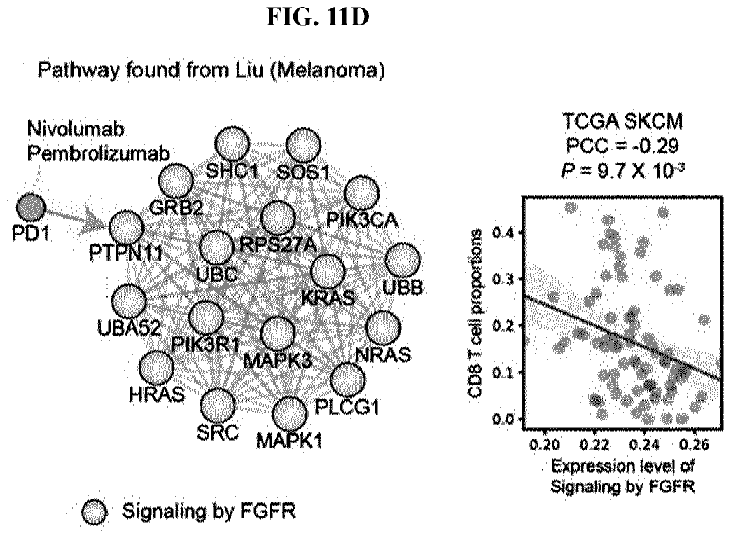

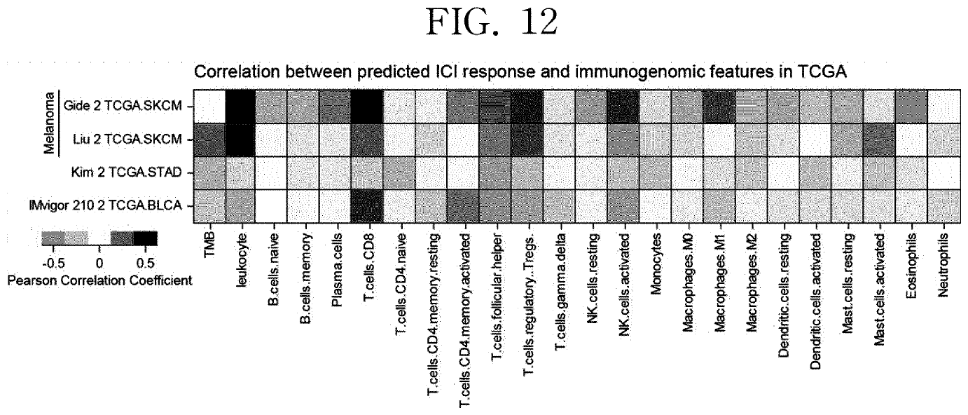

Because NetBio robustly performed the best across distinct cohorts encompassing three different cancer types, whether NetBio-based predictions can recapitulate the immune microenvironment that is associated with immunotherapy responses was investigated. How NetBio-based predictions were correlated with immune contextures in the TCGA datasets (a of FIG. 11) was tested. Specifically, the Gide or Liu dataset (melanoma cohorts) were used to predict ICI responses in melanoma patients in the TCGA dataset (TCGA SKCM), Kim dataset (gastric cancer cohort) to predict TCGA gastric cancer (TCGA STAD), and IMvigor210 dataset (bladder cancer cohort) to predict TCGA bladder cancer (TCGA BLCA) patients and correlated the predicted drug response with (i) the tumor mutation burden (TMB) or (ii) immune contextures of TCGA patients (a of FIG. 11). For immune contextures, immunogenic scores computed by Thorsson et al. were used (Thorsson, V. et al. The Immune Landscape of Cancer. Immunity 48, 812-830.e14 (2018).) The entire correlation results for NetBio-based predictions versus TMB or immune contextures are available in FIG. 12.

NetBio-based predictions successfully recapitulated the immune microenvironments. it was speculated that the correlation results from Gide and Liu cohorts have common characteristics because they both concern melanoma patients. As expected, they exhibited similar immune microenvironment characteristics, including a high positive correlation with leukocyte fractions and CD8 T cell proportions, and a high negative correlation with M2 macrophage proportions (FIGS. 11A-11D).

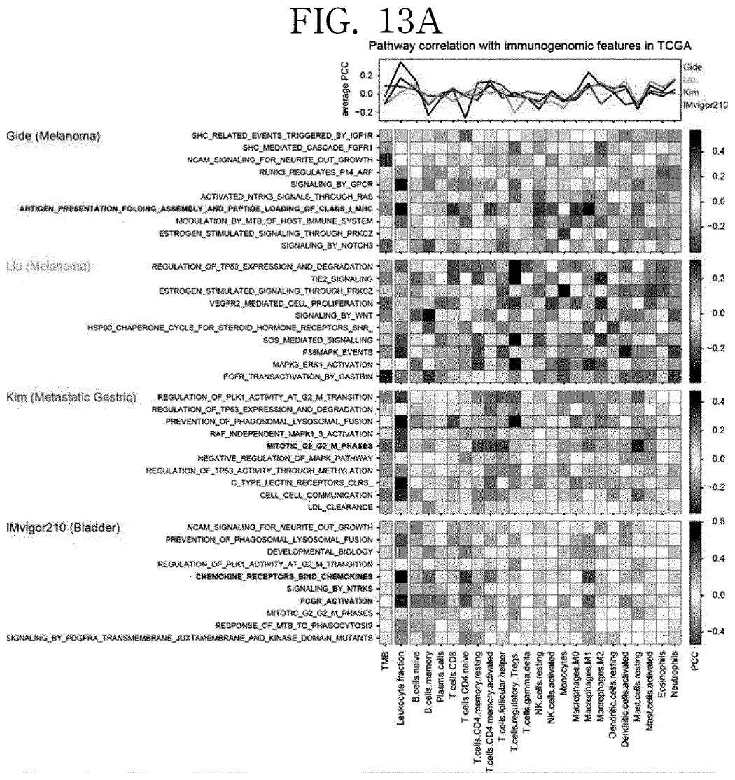

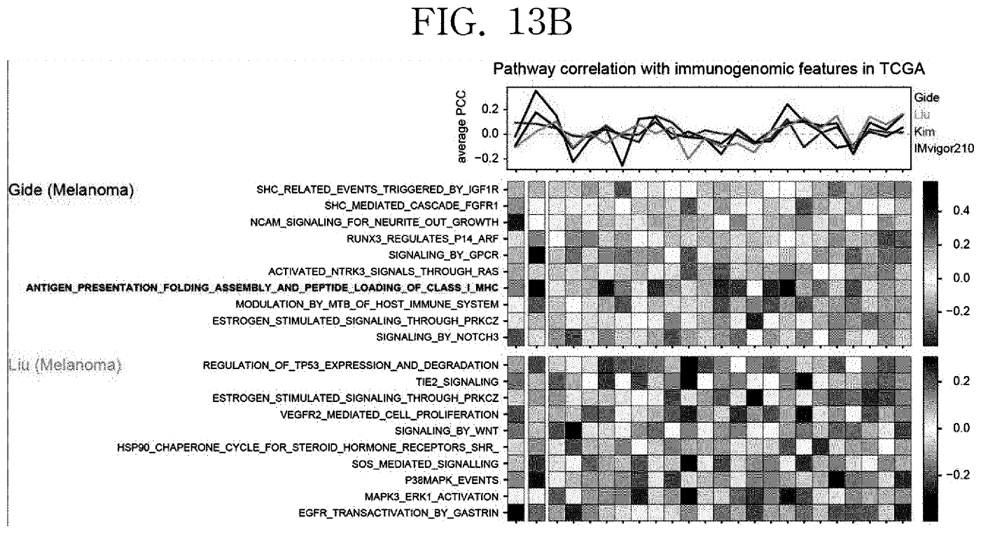

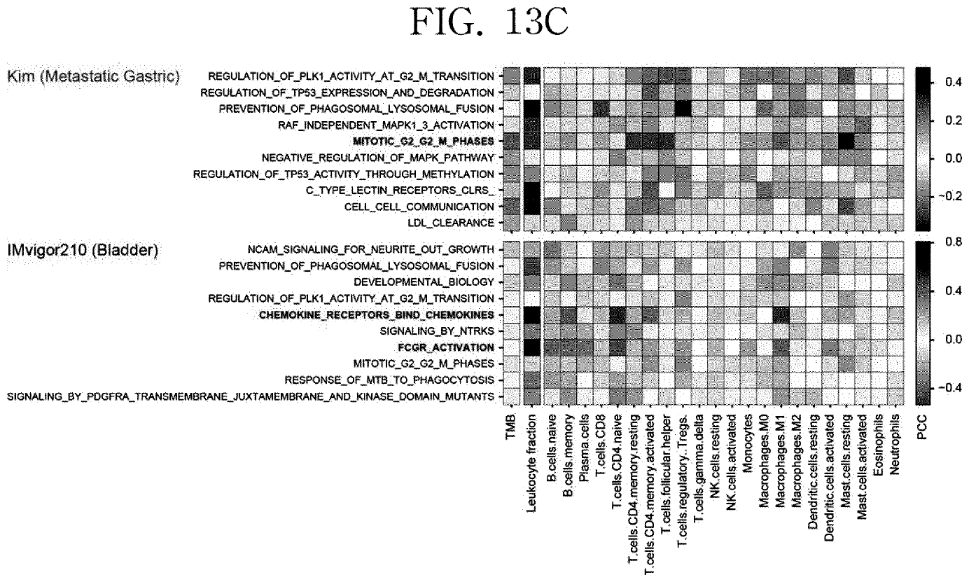

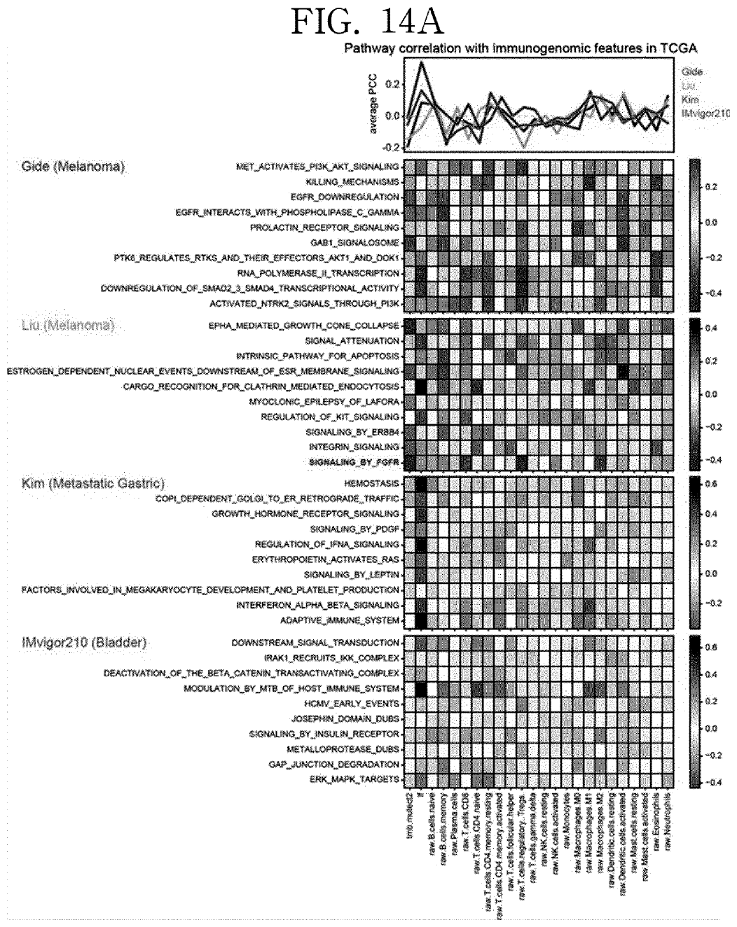

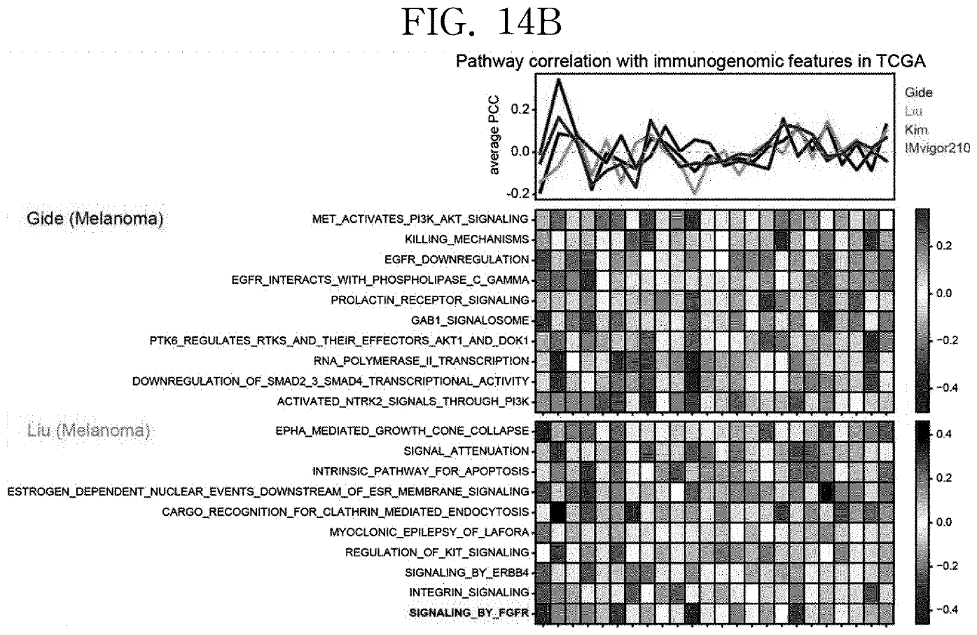

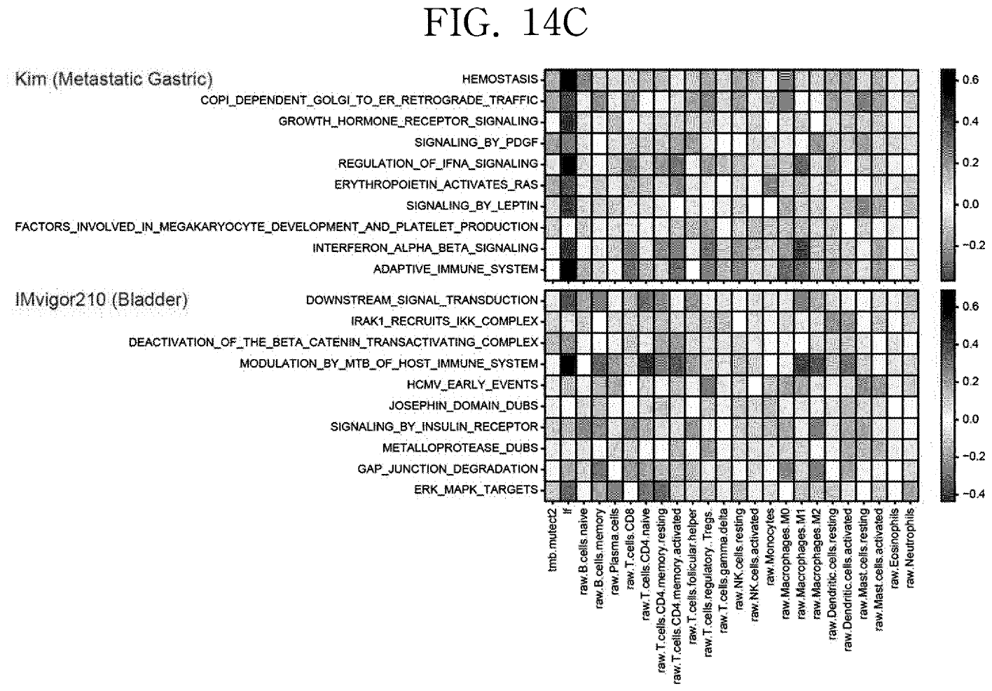

It is further investigated which NetBio pathway was responsible for the high correlation with immune cell proportions. The pathway features of greatest importance from ML training (top 10 greatest feature importance with positive coefficient) using the Gide dataset revealed that ‘antigen presentation folding assembly and peptide loading of class I MHC’ displayed the highest positive correlation with CD8 T cell proportions (c of FIG. 11, FIGS. 13A-13C; PCC=0.41). This finding was expected because antigen presentation by antigen-presenting cells or tumor cells induces the infiltration of CD8 T cells. When using the Liu dataset, among pathways of greatest importance (top 10 greatest feature importance with negative coefficient), ‘FGFR signaling’ showed the highest correlation with CD8 T cell proportions (FIGS. 14A-14C), where the expression level of the pathway was negatively correlated with the cell proportions (c of FIG. 11; PCC=−0.29) Consistent with the findings, recent studies have suggested that fibroblast growth factor 2 depletion can lead to increased T cell recruitment, enabling tumor regression. The results here suggest the following: (i) non-identical CD8 T cell recruitment mechanisms may exist in melanoma and (ii) NetBio can robustly capture CD8 T cell recruitment in tumor samples, even when different melanoma cancer cohorts are used to train an ML model.

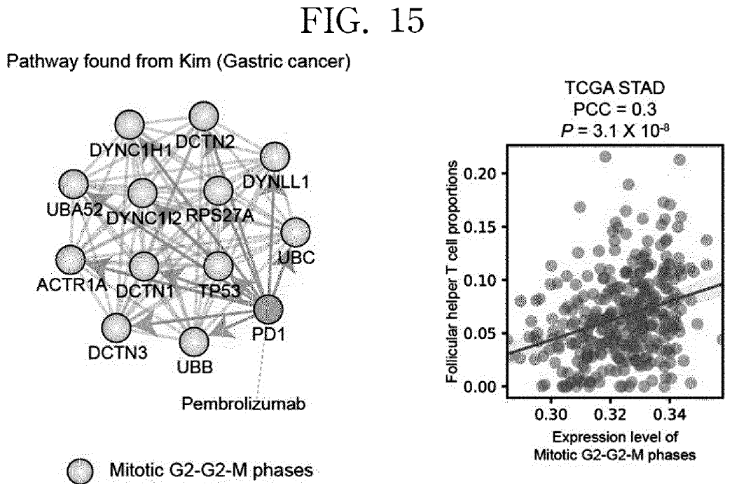

NetBio pathways were also identified that were consistent with the immune microenvironment in gastric and bladder cancer. In gastric cancer, NetBio-based predictions were highly correlated with follicular helper T cell proportions (b of FIG. 11). Among pathways of greatest importance from the Kim cohort, a high expression level of mitotic G2-G2-M phases: was associated with high follicular helper T cell proportions (FIGS. 13A-13C, FIG. 15). Consistent with the results, a previous study reported that the differentiation of helper T cells was regulated by the cell cycle pathway.

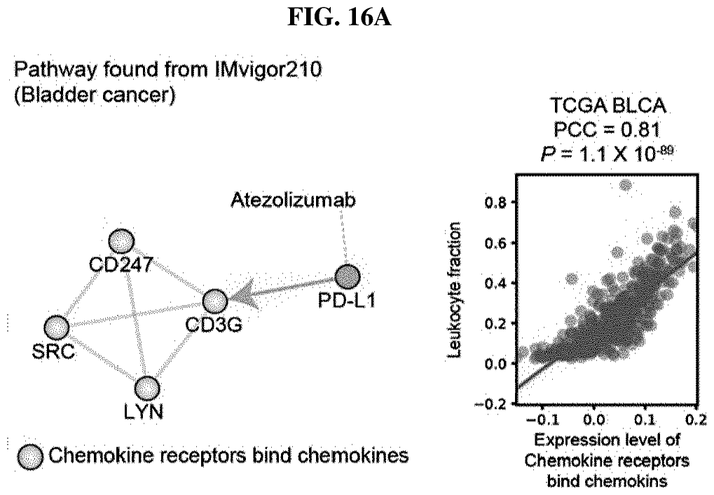

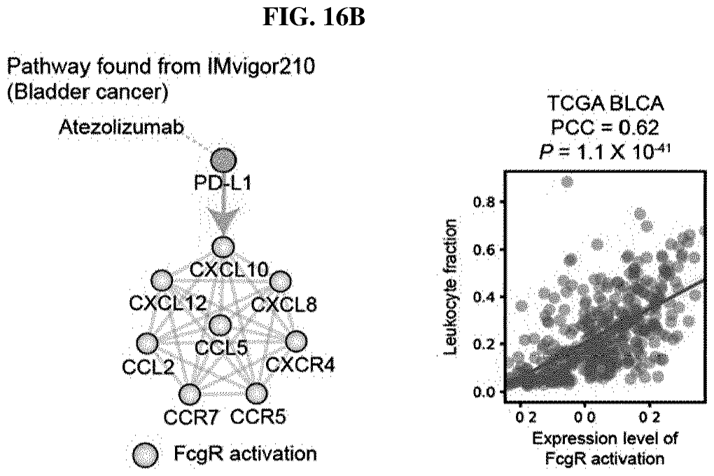

In bladder cancer, it was found that NetBio-based predictions were positively correlated with the leukocyte fractions (b of FIG. 11). Accordingly, the NetBio pathways demonstrated chemotaxis (i.e., chemokine receptors bind chemokines) and phagocytosis (i.e., FcgR activation), which are functions closely associated with immune infiltration (a, b of FIG. 16; PCC>0.6). The results suggest that the immune microenvironments can be captured using NetBio pathways in gastric cancer and bladder cancer.

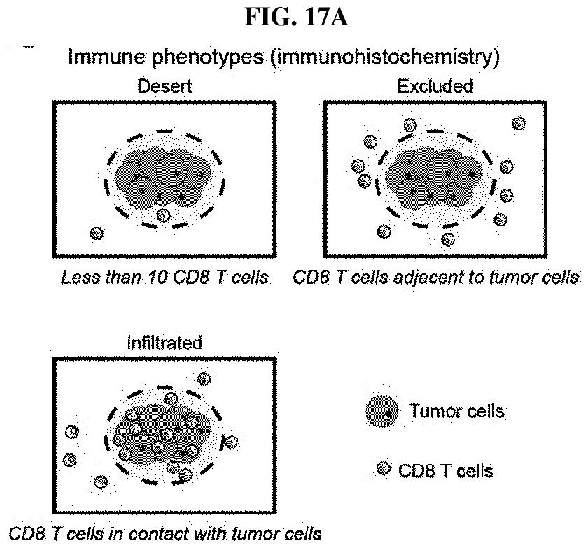

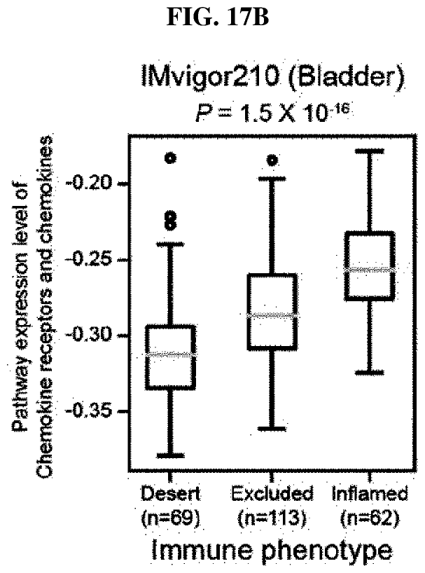

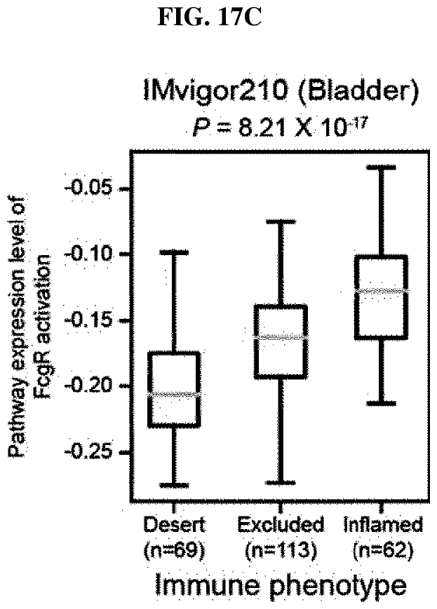

It is further validated that both chemotaxis and phagocytosis pathways (e.g., chemokine receptors bind chemokines and FcgR activation, respectively) are associated with immune infiltration in the PD-L1 treated bladder cancer cohort, using additional immunohistochemistry-based results. Immune phenotypes in the IMvigor210 dataset were used. Specifically, distinct immune phenotypes were used including (i) immune desert (fewer than 10 CD8 T cells), (ii) excluded (CD8 T cells adjacent to tumor cells), and (iii) infiltrated (CD8 T cells in contact with tumor cells) phenotypes (a of FIG. 17) and compared the expression levels of chemotaxis and phagocytosis pathways with the immune phenotypes (b, c of FIG. 17). The immune infiltrated phenotype displayed the highest expression level of the pathways compared with the immune desert or excluded phenotypes (b of FIG. 17; ANOVA P-value <10⁻¹⁶), suggesting that the NetBio pathways can capture leukocyte infiltration fractions in bladder cancer.

Altogether, the results suggest that NetBio can consistently unveil pathways related to the immunotherapy response-associated immune microenvironment.

### Example 7. Combining of TMB and NetBio

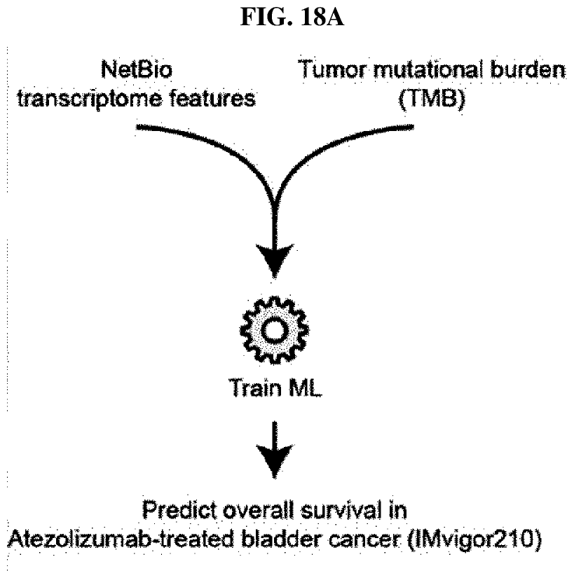

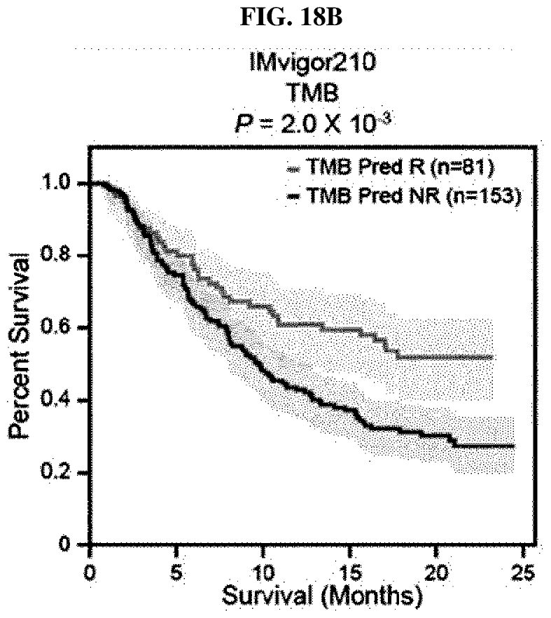

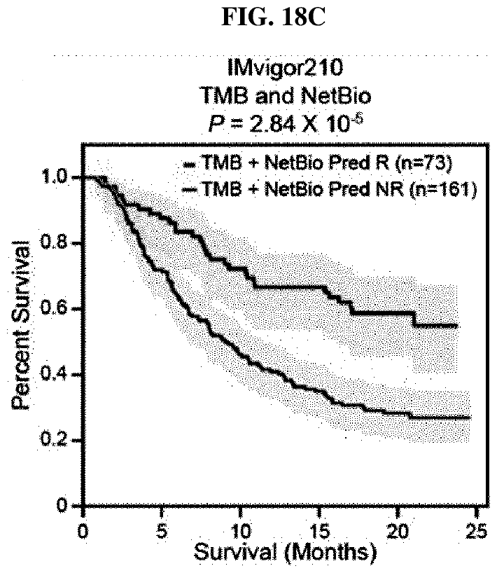

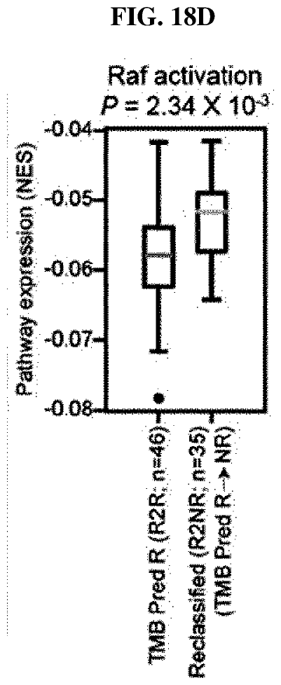

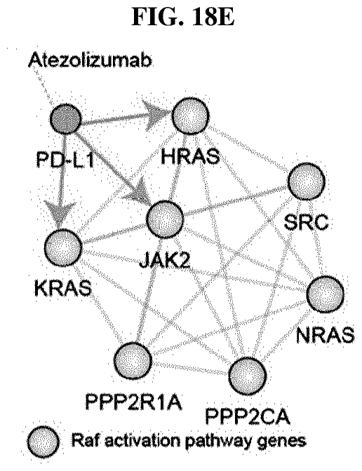

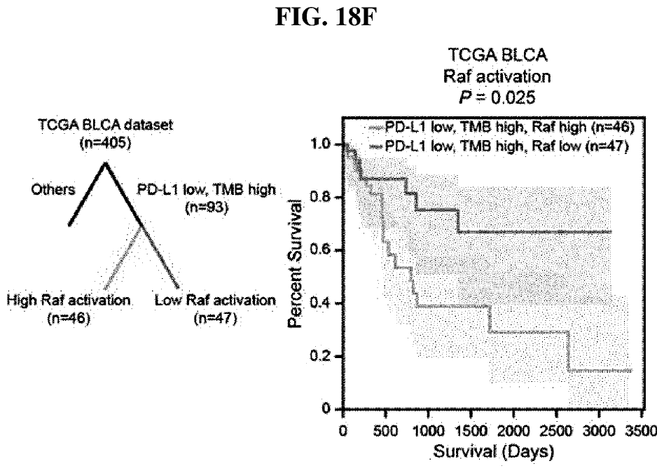

Although a high TMB level is associated with increased benefits of ICI treatment, TMB alone is not a sufficient predictor of the ICI response. Thus, it was tested whether combining the NetBio with TMB-based predictors improves prediction performance (a of FIG. 18). Combining the NetBio expression levels and TMB improved the prediction of the overall survival in bladder cancer patients treated with atezolizumab, which is a PD-L1 inhibitor (b, c of FIG. 18). Using LOOCV to predict the ICI treatment response with only the TMB to train the ML model, the 1-year percent survival difference between the predicted responder group and predicted non-responder group was 18% (b of FIG. 18; log-rank test P=2.0×10⁻³; the 1-year percent survival rates for the predicted responder and predicted non-responder group was 60.8% and 42.8%, respectively). The 1-year percent survival difference was increased to 25.7% when using both the TMB and NetBio (c of FIG. 18; the 1-year percent survival rates for the predicted responder and predicted non-responder group were 66.7% and 40.9%, respectively), as well as improvements in log-rank test statistics (P=2.84×10⁻⁵).

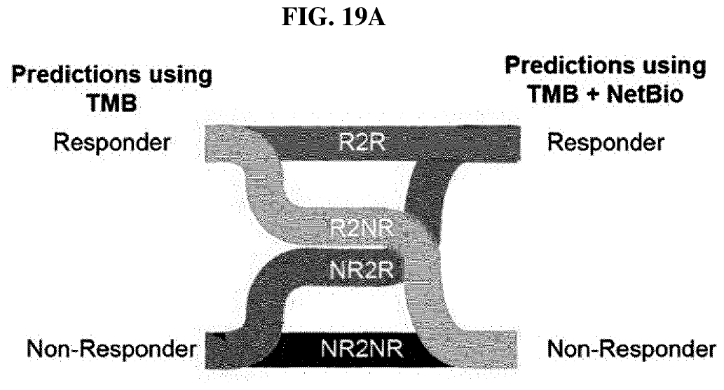

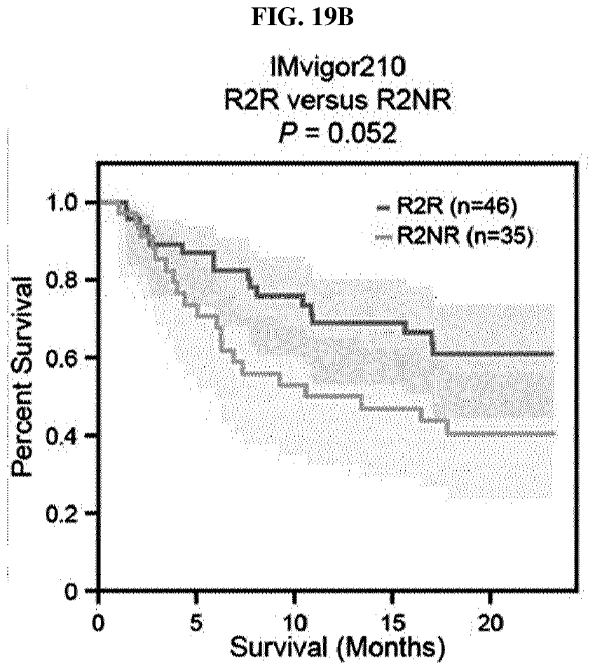

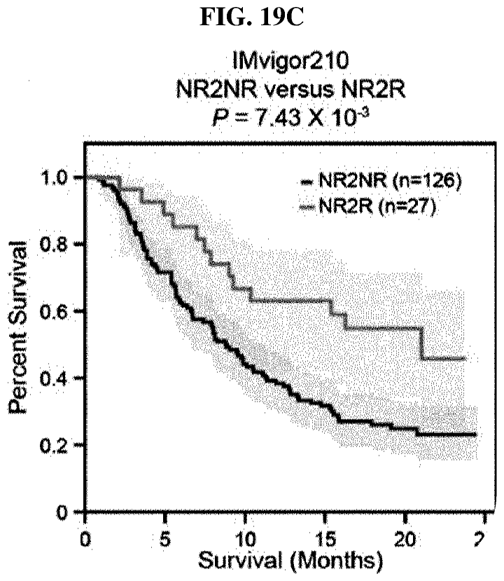

Next, it is observed that the combined predictors correctly reclassified non-responders from predicted responders using TMB alone (NR2R; FIGS. 19A-19C) and correctly reclassified responders from predicted non-responders from TMB-alone predictions (R2NR; FIGS. 19A-19C). R2NR patients exhibited a lower overall survival than the predicted responder group when using only the TMB; the 1-year percent survival decreased to 50% (log-rank test P-value=0.052) Similarly, the 1-year percent survival increased to 63% in NR2R patients and displayed a statistically significant increase in the overall survival compared with the predicted non-responders using TMB-based predictions (c of FIG. 19; log-rank test P-value=7.43×10⁻³) Altogether, the results suggest that TMB combined with NetBio transcriptomic features can improve the correct classification of responders and non-responders.

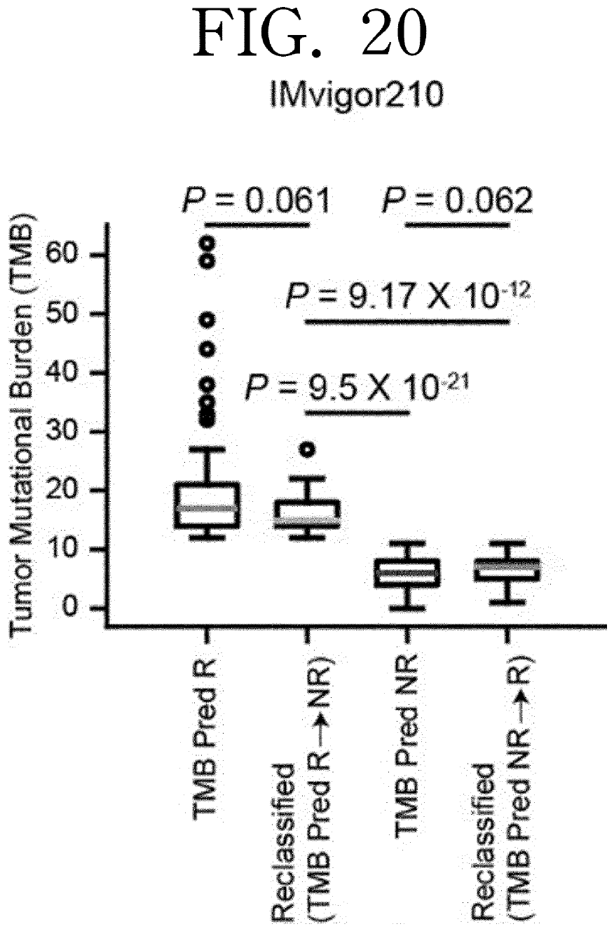

Having observed improved prediction performances, it was sought to identify a feature responsible for the improvements in the prediction performance. It was first observed that the TMB levels remained similar in the reclassified subgroups (FIG. 20), suggesting that the TMB levels are not a confounding factor in the improved prediction of the overall survival. To identify a transcriptomic feature associated with resistance to immunotherapy in the high TMB group, differentially expressed pathways between predicted responders using TMB-based predictions (i.e., high TMB group) and the R2NR group were investigated. The Raf activation pathway was most significantly differentially expressed between the two subgroups (FIG. 18d, two-sided Student t-test P-value=2.34×10⁻³). In detail, patients who were predicted as non-responders from the combined prediction model (i.e., R2NR patients) displayed higher expression of raf activation pathway components. From the PPI network, components of the raf activation pathway, including HRAS, KRAS, and JAK2, were direct neighbors of PD-L1 (FIG. 18e), suggesting that this pathway may exert a mechanistic effect during drug treatment.

To further examine the potential usefulness of the raf activation pathway as an ICI-treatment biomarker, the association among PD-L1 expression, the TMB and the expression level of raf activation components with the overall survival in an external TCGA bladder cancer dataset (n=405) were analyzed. Specifically, it was tested whether Raf activation affected overall survival when (i) the PD-L1 expression was low, simulating PD-L1 inhibition, and (ii) the TMB level was high. The Raf activation pathway had a statistically significant impact on the overall survival in bladder cancer patients exhibiting low PD-L1 expression and high TMB levels (f of FIG. 18; P-value=0.025). Importantly, higher expression of the Raf activation pathway was associated with poor overall survival, a finding that is consistent with PD-L1 inhibitor-treated patients exhibiting resistance to the treatment (d, f of FIG. 18). Altogether, the results suggest that (i) network-based transcriptomic biomarkers can help improve TMB-based immunotherapy response predictions and (ii) novel ICI response biomarkers can be identified using network-based approaches.

We claim:

1. A device for determining whether an immuno-oncology drug is effective to a cancer patient by a computing device, comprising:
a reactome pathway extraction unit configured to extract a target reactome pathway including a target of the immuno-oncology drug from a genomic network;
a gene activity information conversion unit configured to convert gene activity information from transcriptome data of a target cancer patient, who will undergo cancer immunotherapy with the immuno-oncology drug, into activity information of the target reactome pathway; and
a determination unit configured to determine whether the target cancer patient responds to the immuno-oncology drug by inputting target gene information into a pre-trained the immuno-oncology drug response determination model.

2. The device of claim 1,
wherein the pathway extraction unit detects a target node corresponding to the target and a plurality of proximal nodes close to the target node from the genomic network based on influence scores via network propagation using a page-rank algorithm.

3. The device of claim 2,
wherein the pathway extraction unit selects the target reactome pathway from among a plurality of reactome pathways based on normalized enrichment scores (NES) through a gene set enrichment test and a hypergeometric test.

4. The device of claim 1,
wherein the genomic network is a Protein-Protein Interaction network.

5. The device of claim 1,
wherein the immuno-oncology drug includes at least one of an anti-PD-1 antibody, an anti-PD-L1 antibody, and an anti-CTLA4 antibody.

6. The device of claim 1,
wherein the target includes at least one of a PD-1 protein, a PD-L1 protein, and a CTLA4 protein.

7. The device of claim 1,
wherein the immuno-oncology drug response determination model is pre-trained based on the target gene information of a plurality of cancer patients and clinical outcomes on the presence or absence of response to the immuno-oncology drug.

8. A method for determining whether an immuno-oncology drug is effective to a cancer patient by a computing device, comprising:
a process of extracting a target reactome pathway including a target of the immuno-oncology drug from a genomic network;
a process of converting gene activity information from transcriptome data of a target cancer patient, who will undergo cancer immunotherapy with the immuno-oncology drug, into activity information of the target reactome pathway; and
a process of determining whether the target cancer patient responds to the immuno-oncology drug by inputting target gene information into a pre-trained immuno-oncology drug response determination model.

9. The method of claim 8,
wherein the process of extracting the target reactome pathway includes:
a process of detecting a target node corresponding to the target and a plurality of proximal nodes close to the target node from the genomic network based on influence scores via network propagation using a page-rank algorithm.

10. The method claim 9,
wherein the process of extracting the target reactome pathway further includes:
a process of selecting the target reactome pathway from among a plurality of reactome pathways based on normalized enrichment scores (NES) through a gene set enrichment test and a hypergeometric test.

11. The method claim 8,
wherein the genomic network is a Protein-Protein Interaction network.

12. The method of claim 8,
wherein the immuno-oncology drug includes at least one of an anti-PD-1 antibody, an anti-PD-L1 antibody, and an anti-CTLA4 antibody.

13. The method of claim 8,
wherein the target includes at least one of a PD-1 protein, a PD-L1 protein, and a CTLA4 protein.

14. The method of claim 8, further comprising:
a process of training the immuno-oncology drug response determination model based on the target gene information of a plurality of cancer patients and clinical outcomes on the presence or absence of response to the immuno-oncology drug.
